(19) United States
(12) Patent Application Publication
(10) Pub. No.: US 2020/0061092 A1
(43) Pub. Date: Feb. 27, 2020

(54) METHODS AND COMPOSITIONS FOR TREATING DISORDERS ASSOCIATED WITH MUSCLE WEAKNESS

(71) Applicant: The Charlotte Mecklenburg Hospital Authority d/b/a Atrium Health, Charlotte, NC (US)

(72) Inventors: Qi Long Lu, Charlotte, NC (US); Marcela Cataldi, Harrisburg, NC (US); Pei Juan Lu, Charlotte, NC (US); George McLendon, Davidson, NC (US)

(21) Appl. No.: 16/549,986

(22) Filed: Aug. 23, 2019

Related U.S. Application Data
(60) Provisional application No. 62/722,709, filed on Aug. 24, 2018.

(51) Int. Cl. A61K 31/7004; A61K 9/00; A61K 9/20; A61K 47/38; A61P 21/00

(52) U.S. Cl. CPC A61K 31/7004; A61K 9/0004; A61P 21/00; A61K 47/38; A61K 9/0053; A61K 9/20

(57) ABSTRACT

The present invention provides a method of treating a disorder associated with muscle weakness in a subject, comprising administering to the subject a controlled-release composition comprising an effective amount of ribitol and/or ribose, thereby treating the disorder associated with muscle weakness.

### STATEMENT OF PRIORITY

This application claims the benefit, under 35 U.S.C. § 119(e), of U.S. Provisional Application No. 62/722,709, filed Aug. 24, 2018, the entire contents of which are incorporated by reference herein.

### FIELD OF THE INVENTION

The present invention provides methods to treat a disorder associated with muscle weakness or inhibit the development of muscle weakness in a subject.

### BACKGROUND OF THE INVENTION

O-mannosylation of alpha dystroglycan (α-DG), specifically the synthesis of laminin-binding matriglycan (F-α-DG) is conserved at least in vertebrates and critical for neuronal development and muscle integrity and functions. F-α-DG also acts as viral receptors and plays prominent roles in epithelium adhesion and signaling. Hypoglycosylation is involved in cancer development and progression and underlie specific types of muscular dystrophy, in particular dystroglycanopathy with and without defects in neuronal development. One most common dystroglycanopathy caused by mutations in the FKRP gene manifests a wide range of disease severity from mild limb girdle muscular dystrophy (LGMD) 21 to severe congenital muscular dystrophy (CMD), Walker-Warburg syndrome, and muscle-eye-brain disease. Lack of F-α-DG results in progressive degeneration of both skeletal and cardiac muscles. Consequently, patients gradually lose mobility with impaired and ultimately failure of respiratory and cardiac functions. The severe forms of the disease can affect central nerve and optical systems with developmental delay and mental retardation. Currently no treatment is available although several experimental therapies are being tested pre-clinically.

Alpha-DG is a peripheral membrane protein extensively glycosylated with both N- and O-linked glycans, the latter acting as a cellular receptor for laminin and other extracellular matrix (ECM) proteins, including agrin, perlecan, neurexin and pikachurin. The interaction of α-DG with ECM proteins is critical for maintaining muscle integrity. The structure of the laminin-binding O-mannosylated glycan on α-DG (F-α-DG) has recently been delineated with the following chain: (3GlcA-β1-3Xyl-α1) n-3GlcA-β1-4Xyl-Rbo5P-1Rbo5P-3GalNAc-β1-3GlcNAc-β1-4(P-6) Man-1-Thr/ser. The glycan chain extension pathway is completed by the following proposed transferase activity: POMT1 and POMT2 catalyze the initial O-mannosylation of the proteins. Further extension of the sugar chain is carried out by POMGnT2 (GTDC2), B3GALNT2, FKTN, FKRP, TMEM5 and B4GAT1 successively. Finally, LARGE acts as a bifunctional glycosyltransferase having both xylosyltransferase and glucuronyltransferase activities, producing repeated units of 3 GlcA-1-3Xyl-1.

The advances in unraveling the pathway for F-α-DG open new venues for experimental therapy. Recently isoprenoid synthase domain containing (ISPD) has been identified as a cytidyltransferase (pyrophosphorylase) producing CDP-ribitol. Furthermore, CDP-ribitol has now been confirmed by several groups as the substrate of FKRP and FKTN for the extension of the glycan chain of α-DG with ribitol-5-phosphate (ribitol-5P). Interestingly, a study from Gerin et al. demonstrated that ribitol treatment of HEK293 cells overexpressing ISPD and patient-derived ISPD-deficient fibroblasts leads to an increase of CDP-ribitol levels and partially corrects the defect in F-α-DG caused by loss of ISPD function. It is also noted that overexpression of ISPD increased ribitol incorporation into α-DG in wild-type cells, suggesting that the levels of CDP-ribitol might be a limiting factor of this O-mannosylation. These results raise one intriguing possibility: if conversion of ribitol to CDP-ribitol is not a rate-limiting process in muscles in the FKRP mutant mice in vivo as suggested in the normal mice, then an increase in the intracellular levels of ribitol could increase the levels of CDP-ribitol. Since most mutant FKRPs retain at least partial function, an increase in the levels of CDP-ribitol substrate might enhance the efficiency of remaining function of mutant FKRP, thus compensating for the reduced function of mutant FKRPs and enhancing F-α-DG.

The present invention overcomes previous shortcomings in the art by providing pharmaceutical compositions and methods of their use in treating muscular dystrophy and other disorders.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one aspect, the present invention provides a method of treating a disorder associated with muscle weakness in a subject, comprising administering to the subject a controlled-release composition comprising an effective amount of ribitol and/or ribose, thereby treating the disorder associated with muscle weakness.

In an additional aspect, the present invention provides a method of treating or inhibiting the development of muscle weakness in a subject, comprising administering to the subject a controlled-release composition comprising an effective amount of ribitol and/or ribose, thereby treating or inhibiting the development of muscle weakness.

The present invention is explained in greater detail below.

### BRIEF DESCRIPTION OF THE DRAWINGS

FIG. 1: Induction of F-α-DG in cardiac and skeletal muscles by ribitol in P448L mutant mouse treated for 1 month. Four-week-old P448L mice were given drinking water only (n=4), or drinking water supplemented with 5% ribitol (n=4) for 1 month. Immunohistochemical staining with IIH6C4 antibody of cardiac (heart), tibialis anterior (TA), and diaphragm muscles from the untreated and 5% ribitol-treated P448L mice (left and middle panel, respectively), and C57BL/6 control (C57, right panel). Arrows indicate the revertant fibers expressing detectable F-α-DG. Scale bar, 50 μm. Cellular nuclei were counterstained with DAPI.

FIGS. 2a-b: Detection and quantification of ribitol, ribitol-5P and CDP-ribitol by LC/MS-MS (2a) LC/MS-MS detection of ribitol, ribitol-5P and CDP-ribitol from heart and quadriceps of 4-week-old P448L mice treated with drinking water only (untreated) or water supplemented with 5% ribitol for 1 month. (2b) Quantification of ribitol, ribitol-5P and CDP-ribitol levels by LC/MS-MS from heart (H) and quadriceps (Q) of untreated and 5% ribitol-treated P448L mice, and untreated C57 control mice (n=4 for all cohorts). Box represents 25^{th} and 75^{th} percentiles. Line represents median and “+” represents mean. Whiskers extend from min to max value. Unpaired t test *p<0.05.

FIGS. 3a-c: Induction of F-α-DG in cardiac and skeletal muscles of P448L mice treated with ribitol for 6 months. Seven-week-old P448L mice were given drinking water only (n=4), or drinking water supplemented with 5% ribitol (n-4). (3a) IIH6C4 immunohistochemical staining of cardiac (heart), tibialis anterior (TA), and diaphragm tissues from either untreated or 5% ribitol-treated P448L mice (left and middle panel, respectively), and C57 mice. Nuclei were counterstained with DAPI. Arrows indicate the revertant fibers expressing F-α-DG. Arrow head indicates the degenerating fibers and focal accumulation of nuclei. Scale bar, 50 μm. (3b) Western blot and laminin overlay assay of lysates from heart, TA and diaphragm (diaph) of two untreated (−) or two ribitol-treated (+) P448L, and C57 mice. F-α-DG was detected by blotting with IIH6C4 and by laminin overlay assay (Laminin OL). Core of α-DG was detected by AF6868 antibody with weaker signals for the ribitol treated samples. Detection of α-actin was used as loading control. Arrow heads Arrowheads indicate laminin binding bands. The upper band in laminin binding assay is endogenous laminin present in all samples. (3c) Quantification of IIH6C4 band intensity from western blot. Values were normalized to α-actin expression for each tissue and presented as percentage of C57 levels. Error bars represent mean±SEM. Unpaired t test *p≤0.05.

FIGS. 4a-c: Histopathology of muscle tissues from ribitol-treated P448L mice. Seven-week-old P448L mice were given drinking water only, or drinking water supplemented with 5% ribitol for either 3 months (3M) or 6 months (6M). (4a) H&E staining of heart, tibialis anterior (TA), and diaphragm tissues from untreated (Untreated 3M and Untreated 6M) or ribitol-treated (5% ribitol 3M and 5% ribitol 6M) P448L mice, and C57 mice. Arrow indicates areas of heavy infiltration in the control TA muscle. Scale bar, 50 μm. (4b) Fiber size distribution of TA muscles of either untreated (n=3) or ribitol-treated (n=6) P448L mutant mice, and C57 (n=3) mice. (4c) Percentage of CNF in TA muscles of P448L mice treated with 5% ribitol for 3M (n=6) and 6M (n=4), or aged matched untreated (n=3) P448L mice and C57 (n=3) mice. Error bars represent mean±SEM. Unpaired t test *p<0.05.

FIGS. 5a-b: Effect of ribitol treatment on muscle fibrosis in P448L mice. Seven-week-old P448L mice were given drinking water only or drinking water supplemented with 5% ribitol for either 3 months (3M) or 6 months (6M). (5a) Masson's Trichrome staining of heart, tibialis anterior (TA), and diaphragm muscles from untreated (Untreated 3M, and Untreated 6M) or 5% ribitol-treated (5% ribitol 3M and 5% ribitol 6M) P448L mice and C57 mice. Staining represents area of fibrotic tissue. (5b) Percentage of fibrotic areas quantified from Masson's Trichrome staining of 3 months (3M) and 6 months (6M) untreated and 5% ribitol-treated heart, TA, quadriceps (QUAD), and diaphragm (DIAPH) muscles (n=6 for 3M, n=4 for all other cohorts). Error bars represent mean±SEM. Unpaired t test *p<0.05.

FIGS. 6a-c: Induction of F-α-DG in cardiac and skeletal muscles of P448L mice treated with 10% ribitol from pregnancy. P448L breeding females were treated with 10% ribitol in drinking water at onset of pregnancy with pups continuing to receive treatment for 19 weeks. Untreated P448L mice were given drinking water only. (6a) Immunohistochemical staining with IIH6C4 antibody of cardiac (heart), tibialis anterior (TA), and diaphragm muscles from the untreated and 10% ribitol-treated P448L mice (left and middle panel, respectively) and C57 mice. Arrows indicate the degenerating fibers with staining for cytoplasmic Ig. Scale bar, 50 μm. Cellular nuclei were counterstained with DAPI. (6b) Western blot analysis of protein lysates from heart, TA and diaphragm (diaph) of two untreated (−) and two 10% ribitol-treated (+) P448L, and C57 mice. F-α-DG was detected by blotting with IIH6C4 and by laminin overlay assay (Laminin OL). Core of α-DG was detected by AF6868 antibody. Detection of α-actin was used as loading control. (6c) Quantification of IIH6C4 band intensity from western blot. Values were normalized to α-actin expression for each tissue and presented as percentage expression compared to C57. Error bars represent mean±SEM. Unpaired t test *p≤0.05.

FIG. 7a-e: Effect of early 10% ribitol treatment on histopathology and muscle function of P448L mice. Mice were treated from pregnancy to 19 weeks of age. Control P448L mice were given drinking water only. (7a) H&E staining of tibialis anterior (TA) tissues from either untreated or 10% ribitol-treated P448L mice. Arrow indicates the degenerating fibers. Scale bar, 50 μm. Percentage of centrally-nucleated fibers (% CNF) in TA muscles treated with 10% ribitol or aged matched untreated P448L and C57 mice (n=3 for all cohorts). (7b) Masson's Trichrome staining of heart, TA (tibialis anterior) and diaphragm. Staining represents area of fibrosis. Percentage of fibrotic areas quantified from the treated and age-matched untreated P448L and C57 (n=3 for all cohorts) mice. (7c) Treadmill exhaustion test assessing distance (m) and running time (min) in untreated (n=10) or 10% ribitol-treated (n=15) P448L mutant and C57 mice (n=10) at the age of 17 weeks. Unpaired t test *p<0.05. (7d) Grip strength test in untreated (n=10) or 10% ribitol-treated (n=15) P448L and C57 mice (n=10) at the age of 18 weeks. Force (Unite) is normalized to bodyweight (gr). (7e) Respiratory function from untreated (n=10) or 10% ribitol-treated (n=15) P448L and C57 control mice (n=10) at 18 weeks of age. (TV: tidal volume, MV: minute volume, EEP: end-expiratory pause, EIP: end-inspiratory pause). Error bars represent mean±SEM. Unpaired t test *p<0.05.

FIG. 8: Model for ribitol-induced functional glycosylation of α-DG in FKRP mutant cells. ?: mechanism(s) not understood; *: first ribitol-5P on the Core M3 of α-DG is transferred by fukutin using also CDP-ribitol as the donor substrate.

FIGS. 9a-b: LC/MS-MS chromatograms for the detection and quantification of synthetic ribitol, ribitol-5P and CDP-ribitol. (9a) MS-MS Spectrum (upper panels) and chromatograms with retention time (lower panels) of the synthetic ribitol, ribitol-5P and CDP-ribitol. (9b) Standard curve from serial dilution of stock solutions for each metabolite.

FIGS. 10a-b: LC/MS-MS chromatograms for the detection of isotopically labeled ¹³C-ribitol, ¹³C-ribitol-5P and CDP-¹³C-ribitol. (10a) ¹³C-ribitol chromatogram with retention time and MS-MS Spectrum with fragmentation. (10b) LC/MS-MS detection of ¹³C-ribitol, ¹³C-ribitol-5P, CDP-¹³C-ribitol, and their unlabeled analogs from untreated and 5 mM ¹³C-ribitol-treated differentiated C2C12 myotubes in vitro.

FIGS. 11a-b: Induction of F-α-DG in three-month 5% ribitol-treated P448L mutant mice. (11a) Immunohistochemical staining for F-α-DG with IIH6C4 antibody in cardiac (heart), tibialis anterior (TA), and diaphragm muscles from P448L mice drinking either water (P448L untreated) or water supplemented with 5% ribitol (P448L 5% ribitol) and wild-type C57 mice. Arrow indicates the revertant fibers expressing detectable F-α-DG and arrow heads indicate the degenerating fibers. Cellular nuclei were counterstained with DAPI. Scale bar, 50 μm. (11b) Levels of FKRP and LARGE transcripts in cardiac muscle (heart), skeletal muscle (tibialis anterior, TA) and diaphragm (diaph) analyzed by quantitative real-time PCR (n=3). Error bars represent mean±SEM. Unpaired t test, * p<0.05.

FIGS. 12a-c: Effect of 5% ribitol treatment on histopathology of P448L mutant mice. (12a) H&E staining of quadriceps from P448L mutant mice drinking either water (Untreated) or water supplemented with 5% ribitol (5% ribitol). Treatments were maintained for either 3 (3M) or 6 months (6M). Scale bar, 50 μm. (12b) Fiber size distribution from quadriceps of either 5% ribitol treated (n=6) or age-matched untreated (n=3) P448L mutant mice, and wild-type C57 (n=3) mice. (12c) Percentage of centrally-nucleated fibers (% CNF) from quadriceps of 3M (n=6) and 6M (n=4) ribitol-treated or age-matched untreated (n=3) P448L mutant mice, and wild-type C57 mice (n=3). Error bars represent mean±SEM. Unpaired t test *p<0.05.

FIG. 13: Histopathology of diaphragms from 5% ribitol-treated P448L mutant and control mice. H&E staining of diaphragms from two untreated (Untreated 1 and 2) and two 5% ribitol-treated (5% Ribitol 1 and 2) P448L mutant mice. Treatments were maintained for either 3 months (3M) or 6 months (6M). Scale bar, 50 μm.

FIG. 14: Fibrosis in diaphragms of untreated and 5% ribitol-treated P448L mutant mice. Masson's Trichrome staining of diaphragms from two untreated (Untreated 1 and 2) and two 5% ribitol-treated (5% ribitol 1 and 2) P448L mutant mice. Treatments were maintained for either 3 months (3M) or 6 months (6M). Scale bar, 50 μm.

FIGS. 15a-b: Evaluation of respiratory skeletal muscle function in 5% ribitol-treated P448L mutant mice. Seven-week-old P448L mutant mice were given drinking water only, or drinking water supplemented with 5% ribitol for either 3 months (3M) or 6 months (6M). (15a) Respiratory function parameters from untreated (n=10 for 3M and 6M) or 5% ribitol-treated (n=10 for 3M, n=4 for 6M) P448L mice. (TV: tidal volume, EV: expiratory volume, MV: minute volume, PIF: peak inspiratory flow, PEF: peak expiratory flow, and f: breathing frequency). (15b) Treadmill exhaustion test assessing the distance (m, meters) and time (min, minutes) until exhaustion run by untreated (n=10 for 3M and 6M) or 5% ribitol-treated (n=10 for 3M, n=4 for 6M) P448L mutant mice. Error bars represent mean±SEM. Unpaired t test *p<0.05.

FIG. 16: Histopathology in skeletal and cardiac muscles of P448L mutant mice treated with 10% ribitol. P448L mutant mice were treated with 10% ribitol in drinking water when the breeding female became pregnant, and the pups continued to be treated until 19 weeks of age. Untreated P448L mutant mice were given drinking water only. H&E staining of heart and diaphragm tissues from either untreated (Untreated 1 and 2), or 10% ribitol-treated (10% ribitol 1 and 2) P448L mutant mice. Scale bar, 50 μm.

FIGS. 17a-b: Effect of 10% ribitol treatment on respiratory function and body weight of P448L mutant mice. P448L mutant mice were treated with 10% ribitol in drinking water when the breeding female became pregnant, and the pups continued to be treated until 19 weeks of age. Untreated P448L mutant mice were given drinking water only. (17a) Respiratory function parameters from untreated (n=10) or 10% ribitol-treated (n=15) P448L mice, and C57 control mice at 18 weeks of age. (EV: expiratory volume, RT: relaxation time, Penh: enhanced pause). Error bars represent mean±SEM. Unpaired t test *p<0.05. (17b) Body weight (gr) change among the mice treated from the embryonic stage (10% ribitol) or those treated from 7 weeks of age (5% ribitol) in comparison with age-matched untreated P448L mutant mice.

FIGS. 18a-b: Evaluation of ribitol toxicity in kidney, liver, spleen and serum. (18a) H&E staining of kidney, liver and spleen from P448L mice drinking water only (untreated) or water supplemented with 5% ribitol (5% ribitol). Treatment were maintained for 3 (3M) or 6 months (6M). Scale bar, 100 μm (18b) Levels of serum biochemical analytes from P448L females (F) and males (M) mice, either untreated or treated with 5% ribitol for 6 months. (Untreated F, n=8; untreated M, n=9; treated F, n=5; treated M, n=5). (ALP; alkaline phosphatase, ALT: alanine transaminase, TRG: triglycerides, t-Bil: total bilirubin, c-Bil: conjugated bilirubin, unc-Bil: unconjugated bilirubin, BUN: urea, Crea: creatinine, GLU: glucose). Box represents 25^{th} and 75^{th} percentiles. Line represents median. “+” represents mean. Whiskers extend from minimum to maximum value.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

The present invention is based on the unexpected discovery that ribitol and/or ribose in a controlled-release composition can be used to treat a disorder associated with muscle weakness in a subject. Thus, in one embodiment, the present invention provides a method of treating a disorder associated with muscle weakness in a subject, comprising administering to the subject a controlled-release composition comprising an effective amount of ribitol and/or ribose, thereby treating the disorder associated with muscle weakness.

An effective amount of ribitol and/or ribose can be determined, for example, by correlating the amount of ribitol and/or ribose with the efficacy of the treatment on muscle pathology and functions according to methods known in the art.

In some embodiments, the effective amount of ribitol and/or ribose can be in a range from about 40% to about 100% (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93 94 95, 96, 97, 98, 99 or 100%) of the controlled-release composition.

In some embodiments, the effective amount of ribitol and/or ribose can be in a range from about 50% to about 80% of the controlled release composition.

In some embodiments, administering the controlled release composition comprising an effective amount of ribitol and/or ribose can result in a serum level in the subject of ribitol and/or ribose in a range from about 200 ug/L to about 20 mg/L (e.g., about 200 ug/L, 300 ug/L, 400 ug/L, 500 ug/L, 600 ug/L, 700 ug/L, 800 ug/L, 900 ug/L, 0.5 mg/L, 1 mg/L, 2 mg/L, 3 mg/L, 4 mg/L, 5 mg/L, 6 mg/L, 7 mg/L, 8 mg/L, 9 mg/L, 10 mg/L, 1 1 mg/L, 12 mg/L, 13 mg/L, 14 mg/L, 15 mg/L, 16 mg/L, 17 mg/L, 18 mg/L, 19 mg/L or 20 mg/L. An effective stable-status serum level of ribitol and ribose can be established based on the correlation between the efficacy of the treatment on muscle pathology and functions and the serum levels of ribitol and ribose.

In some embodiments, administering the controlled release composition comprising an effective amount of ribitol and/or ribose results in a serum level in the subject of ribitol and/or ribose in a range from about 0.5 mg/L to about 5 mg/L.

In some embodiments, the disorder associated with muscle weakness can be associated with a defect in glycosylation of alpha-DG, including situations without clear understanding of the underlying causes for the defect.

In some embodiments, the disorder associated with muscle weakness is a disorder associated with a mutation or loss of function in a fukutin related protein (FKRP) gene and/or a disorder associated with a defect in glycosylation of alpha-DG in the subject. Nonlimiting examples of a disorder associated with a mutation or loss of function in the FKRP gene include limb-girdle muscular dystrophy type 2i (LGMD2i), Walker-Warburg syndrome (WWS), muscle-eye-brain disease (MEB), congenital muscular dystrophy type 1C (MDC 1C), and any combination thereof.

In some embodiments, the subject can be a carrier of a mutated FKRP gene with or without a defect in glycosylation of alpha-DG.

In additional embodiments, the present invention provides a method of treating or inhibiting the development of muscle weakness in a subject, comprising administering to the subject a controlled-release composition comprising an effective amount of ribitol and/or ribose, thereby treating or inhibiting the development of muscle weakness, e.g., muscle weakness which limits or slows daily activity of the subject.

In embodiments of this invention, the controlled-release composition can be a polymer based controlled release system, a micro-capsulation based controlled release system, an osmotic controlled release oral delivery system (OROS), or any combination thereof.

In some embodiments, the controlled-release composition can be a polymer based controlled release system comprising a cross-linked polymer matrix loaded with an effective amount of ribitol and/or ribose, which, for example, is released from and/or within polymers at a desirable rate.

In some embodiments, the cross-linked polymer matrix can comprise a cellulose based polymer, a non-cellulose based polymer, a natural polymer, an acrylic acid based polymer, or any combination thereof.

In some embodiments, the controlled-release composition can comprise hydroxypropyl methylcellulose (HMPC), methylcellulose, chitosan, hydroxyethyl methacrylate (HEMA), alginate, fibrin, gelatin, collagen, hyaluronic acid, dextran, N-(2-hydroxypropyl)methacrylate (HPMA), N-vinyl-2-pyrrolidone (NVP), N-isopropyl acrylamide (NIPAAm), vinyl acetate (VAc), acrylic acid (AA), methacrylic acid (MAA), microcrystalline cellulose (MCC), polyethylene glycol acrylate/methacrylate (PEGA/PEGMA), polyethylene glycol diacrylate/dimethacrylate (PEGDA/PEGDMA), 2-(dimethylamine)ethyl methacrylate (DMAEMA, polypropylene oxide-polyethylene oxide-polypropylene oxide (PPO-PEO-PPO) block polymers, or any combination thereof.

In some embodiments, the cross-linked polymer matrix comprises hydroxypropyl methylcellulose (HMPC) and microcrystalline cellulose (MCC).

In embodiments of this invention, the controlled-release composition can be encapsulated and/or compressed into a tablet.

In some embodiments, the encapsulated or compressed controlled-release composition can be coated with a suitable film coat, erodible outer layer composition, mucoadhesive outer layer composition, or any combination thereof.

In some embodiments, the erodible outer layer composition can comprise HMPC, ethyl cellulose, PEO, or any combination thereof.

In some embodiments, the mucoadhesive outer layer composition can comprise a carbohydrate polymer.

In some embodiments, the controlled-release composition elutes a therapeutically effective amount of ribose and/or ribitol at an elution rate of about 5-20%/hr with a daily dose from about 0.05 g/Kg to about 1 g/Kg body weight. A therapeutically effective elution rate is the rate at which effective serum levels are maintained, as described herein

In some embodiments, the controlled-release composition elutes a therapeutically effective amount of ribose and/or ribitol at an elution rate of about 5-20%/hr (e.g., 5, 6, 7, 8, 9, 10, 11, 12, 13, 14, 15, 16, 17, 18, 19 or 20%) with a daily dose from about 0.1 g/Kg to about 0.2 g/Kg (e.g., about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25 g/Kg) body weight.

In some embodiments, the therapeutically effective amount of ribitol and/or ribose elutes at a rate to obtain a steady state serum concentration that is from about 0.5 mg/L to about 20 mg/L above normal serum levels. In some embodiments, serum levels of ribitol and/or ribose can be determined according to methods known in the art and normal serum levels can be established for a given subject or population based on known methods.

In some embodiments, the therapeutically effective amount of ribitol and/or ribose elutes at a rate to obtain a steady state serum concentration that is from about 1 mg/L to about 5 mg/L (e.g., 1, 2, 3, 4, or 5 mg/L) above normal serum levels.

In some embodiments, a single administration of the controlled-release composition can provide a therapeutically effective steady state serum concentration of ribitol and/or ribose for about 2 hours to about 24 hours (e.g., 2, 3, 4, 5, 6, 7, 8, 9, 10, 11, 12, 13, 14, 15, 16, 17, 18, 19, 20, 21, 22, 23, or 24 hrs).

In some embodiments, a single administration of the controlled-release composition can provide a therapeutically effective steady state serum concentration of ribitol and/or ribose for about 6 hours to about 12 hours.

In some embodiments, the effective amount of ribitol and/or ribose administered to the subject over 24 hours can be about 0.05 g/kg to about 1 g/kg (e.g., 0.05, 0.06, 0.07, 0.08, 0.09, 0.10, 0.11, 0.12, 0.13, 0.14, 0.15, 0.16, 0.17, 0.18, 0.19, 0.20, 0.25, 0.30, 0.35, 0.40, 0.45, 0.50, 0.60, 0.70, 0.80, 0.90, or 1.0 g/kg), based on the body weight of the subject.

In some embodiments, the effective amount of ribitol and/or ribose administered to the subject over 24 hours can be about 0.1 g/kg to about 0.2 g/kg, based on the body weight of the subject.

In some embodiments, the controlled-release composition can be administered orally. In some embodiments, the controlled-release composition can be administered 1, 2, 3, 4, 5, 6, 7, 8, 9, or 10 times daily. In specific embodiments, the controlled-release composition is administered 1, 2, 3, or 4 times daily.

In some embodiments, the controlled-release composition can further comprise pharmaceutically acceptable excipients, diluents, and/or carriers, including, but not limited to glucose, polyethylene glycol (PEG), glycerin, etc.

In some embodiments, the controlled-release composition can further comprise a therapeutic agent. Nonlimiting examples of a therapeutic agent include Tamoxifen, raloxifene, a phosphodiesterase type 5 (PDE5) inhibitor, anti-inflammatory agents, and any combination thereof.

In some embodiments, the controlled release composition can be administered in combination with one or more therapeutic agents for treating and/or inhibiting muscle weakness. In some embodiments the controlled-release composition can be administered or delivered to a subject in combination with (e.g., simultaneously, before and/or after) CTP and/or any other nucleotide in an amount effective for enhancing the effect of the controlled release composition on glycosylation of α-DG or other proteins. Furthermore, the controlled release composition can administered with any other therapy (simultaneously, before and/or after), such as steroid therapy and/or FKRP gene therapy to enhance or increase the therapeutic effect.

In some embodiments, the one or more therapeutic agents can comprise one or more gene therapeutic agents for treating and/or inhibiting the development of muscle weakness in a subject that is a carrier of a mutated FKRP gene with or without a defect in glycosylation of alpha-DG and/or for treating a subject having a disorder associated with a mutation or loss of function in a fukutin related protein (FKRP) gene.

The present invention also provides a method of treating muscular dystrophy without defects in dystroglycan-related genes (e.g., a muscular dystrophy that is not associated with a defect in glycosylation of α-DG) or defects or abnormalities in levels of the ribitol and CDP-ribitol in a subject, comprising administering to the subject an effective amount of a ribitol and/or ribose in the controlled release composition, thereby treating the muscular dystrophy in the subject.

In an additional embodiment, the present invention provides a method of reducing the incidence of a neuronal migration abnormality or other disorder or symptoms associated with a mutation in a FKRP gene or without defect in a dystroglycan-related gene or in glycosylation of α-DG, comprising administering to the mother of the subject, during the subject's gestation in the mother's uterus, an effective amount of ribitol and/or ribose in the controlled release composition, thereby reducing the incidence of a neuronal migration abnormality, or other disorder or symptoms associated with a mutation in the FKRP gene of the subject.

Additionally, the present invention provides a method of treating and/or inhibiting the development of muscle weakness in a subject in need thereof, which can include but is not limited to weakness of skeletal muscle, cardiac muscle and/or respiratory muscle, in any combination, comprising administering to the subject an effective amount of an active agent or composition of this invention.

The methods of this invention can also be used to treat non-muscular dystrophy diseases for which restoration of and/or enhanced glycosylation of α-DG would be beneficial and/or therapeutic.

In some embodiments of the methods of this invention, nonlimiting examples of a disorder associated with a mutation in, or loss of function of, the FKRP gene include limb-girdle muscular dystrophy (LGMD2I), Walker-Warburg syndrome (WWS), muscle-eye-brain disease (MEB), congenital muscular dystrophy type 1C (MDC1C), any other disorder associated with a mutation in, or loss of function of, the FKRP gene, and any combination thereof.

In some embodiments, an active compound or agent for use in the compositions and methods described herein can be ribitol, CDP-ribitol, ribose and/or ribulose.

In the methods of this invention, the ribitol can be, but is not limited to, ribitol (adonitol) pentose alcohol, with or without modifications such as tri-acetylated ribitol (Ribitol(OAc)₃, per-acetylated ribitol (Ribitol(OAc)₅, a precursor thereof, such as ribose, a polysaccharide thereof, a phosphate form thereof, a non-phosphated form thereof, any precursor of a phosphate form, such as Ribose-5-P, any nucleotide form of ribitol (e.g., a nucleotide-alditol having cytosine or other bases as the nucleobase with 1, 2 or 3 phosphate groups and ribitol as the alditol portion), such as CDP-ribitol, CDP-ribitol-OAc2 and any combination or derivative or modification thereof.

The active compound or agent of this invention can be present in a pharmaceutical formulation that comprises substances and/or agents that are not natural products. As a nonlimiting example, the active compound of this invention can be present in a pharmaceutical composition with polyethylene glycol (PEG), which in some embodiments can have a molecular weight (MW) in a range of about 200 to about 500. In some embodiments, a pharmaceutical composition of this invention can comprise glucose.

In some embodiments, the active compound of this invention can comprise a polyalkylene glycol moiety coupled or linked thereto. “Polyalkylene glycol” means straight or branched polyalkylene glycol polymers including, but not limited to, polyethylene glycol (PEG), polypropylene glycol (PPG), and polybutylene glycol (PBG), as well as co-polymers of PEG, PPG and PBG in any combination, and includes the monoalkylether of the polyalkylene glycol. Thus, in various embodiments of this invention, the polyalkylene glycol in the compositions of this invention can be, but is not limited to, polyethylene glycol, polypropylene glycol, polybutylene glycol, and any combination thereof.

In certain embodiments, the polyalkylene glycol of the composition is polyethylene glycol or “PEG.” The term “PEG subunit” refers to a single polyethylene glycol unit, i.e., —(CH₂CH₂O)—. Thus, the active compound can be “pegylated.” In some embodiments, the PEG can have a molecular weight from about 10,000 g/mol to about 30,000 g/mol.

In some embodiments, the polyalkylene glycol (e.g., PEG) can be non-polydispersed, monodispersed, substantially monodispersed, purely monodispersed, or substantially purely monodispersed.

“Monodispersed” is used to describe a mixture of compounds wherein about 100 percent of the compounds in the mixture have the same molecular weight.

“Substantially monodispersed” is used to describe a mixture of compounds wherein at least about 95 percent of the compounds in the mixture have the same molecular weight.

“Purely monodispersed” is used to describe a mixture of compounds wherein about 100 percent of the compounds in the mixture have the same molecular weight and have the same molecular structure. Thus, a purely monodispersed mixture is a monodispersed mixture, but a monodispersed mixture is not necessarily a purely monodispersed mixture.

“Substantially purely monodispersed” is used to describe a mixture of compounds wherein at least about 95 percent of the compounds in the mixture have the same molecular weight and have the same molecular structure. Thus, a substantially purely monodispersed mixture is a substantially monodispersed mixture, but a substantially monodispersed mixture is not necessarily a substantially purely monodispersed mixture.

In some embodiments of the methods of this invention, the active agent can be administered or delivered to a subject in combination with (e.g., simultaneously, before and/or after) CTP and/or any other nucleotide in an amount effective for enhancing the effect of ribitol on glycosylation of α-DG or other proteins. Furthermore, in the methods of this invention, the active agent can administered with any other therapy (simultaneously, before and/or after), such as steroid therapy and/or FKRP gene therapy to enhance or increase the therapeutic effect.

Further aspects of this invention include the use of an active agent of this invention and/or a composition of this invention in the preparation of a medicament for carrying out the methods of this invention.

An additional aspect is the use of an active agent of this invention and/or a composition of this invention for carrying out the methods of this invention.

The ribitol of this invention can be in a composition comprising a pharmaceutically acceptable carrier. The therapeutically effective amount or dosage of ribitol of this invention will vary depending on the subject's condition and therapeutic need, and will also depend, among other things, upon the effect or result to be achieved, the status of the subject and/or the route and/or mode of delivery. In some embodiments, ribitol or any other form(s) that can be converted to ribitol, or ribitol phosphate, or nucleotide-ribitol can be delivered orally in drinking water containing from about 0.1 to about 100% concentration of the drug as many times as desirable, e.g., from about 1 time to about 100 times a day. The drug can also be taken as pellet about 1 to about 10 times daily. The total amount of the drug for daily use can be from about 0.001 g to about 500 g depending on the nature and formulation of the drug, the ribitol or modified ribitol with enhanced effect, etc. The drug can be mixed or combined with any substance for improved delivery, absorption, etc.

Ribitols form in many plants and especially in the plant, Adonis vernalis, also known as spring pheasant's eye, or false hellebore, or yellow pheasant's eye and others. Adonis vernalis belongs to the buttercup family Ranunculaceae. Plants containing ribitols can be administered as the drug for treating FKRP-related diseases and subjects with FKRP mutation and other diseases. Such plants can be directly used as a food supplement, and/or ribitol can be extracted from the plants for administration as described herein.

Administration of the compound or composition of this invention may be by any suitable route, including but not limited to intrathecal injection, subcutaneous, cutaneous, oral, intravenous, intraperitoneal, intramuscular injection, intra-arterial, intratumoral or any intratissue injection, nasal, oral, sublingual, via inhalation, in an implant, in a matrix, in a gel, or any combination thereof.

In further embodiments, the present invention provides a method of enhancing expression of functional glycosylation of alpha-DG in a subject in need thereof, comprising administering to the subject an effective amount of an active agent and/or composition of this invention. An example of a subject in need of such enhancement can be a subject that has muscle weakness without a defect in a gene known to be involved in glycosylation.

The present invention further provides a method of treating a disorder associated with a defect in glycosylation of alpha-DG, comprising administering to a subject that has or is suspected of having a disorder associated with a defect in glycosylation of alpha-DG an effective amount of an active agent and/or composition of this invention. A subject can be suspected of having a defect in glycosylation of alpha-DG if the subject has muscle weakness even in cases where genetic and biochemical analyses of the subject have failed to identify a causative gene defect.

In additional embodiments, the present invention provides a method of treating a disorder associated with muscle weakness, comprising administering to a subject that has or is suspected of having of developing a disorder associated with muscle weakness an effective amount of an active agent and/or composition of this invention. Muscle weakness can imply that a subject is not able to perform the daily activities that a normal person of similar gender, age and other conditions would be expected to be capable of performing An example is the loss of or lack of ability to climb stairs, run or hold an object for an extended period.

Further provided herein is a method of treating a disorder associated with a defect in glycosylation of alpha-DG caused by a mutation in the FKRP gene, comprising administering to a subject that has or is suspected of having a mutation in the FKRP gene an effective amount of an active agent and/or composition of this invention. A mutation in an FKRP gene can be identified by genetic analysis of the nucleic acid of a subject.

### Definitions

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a fatty acid) and the like, is meant to encompass variations of +20%, +10%, ±5%, +1%, ±0.5%, or even±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

“Subject” as used herein includes any animal in which functional glycosylation of alpha-dystroglycan (α-DG) or other proteins is necessary or desired. In some embodiments, the subject is any animal that can receive a beneficial and/or therapeutic effect from restoration of functional glycosylation of alpha-dystroglycan (α-DG) and/or enhancement of glycosylation of α-DG. In some embodiments, the subject is a mammal and in particular embodiments, the subject is a human of any age, race, gender, or ethnicity, etc.

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay or inhibition in the progression of the disease or disorder.

“Treat,” “treating” or “treatment” as used herein also refers to any type of action or administration that imparts a benefit to a subject that has a disease or disorder, including improvement in the condition of the patient (e.g., reduction or amelioration of one or more symptoms), healing, etc.

The terms “therapeutically effective amount,” “treatment effective amount” and “effective amount” as used herein are synonymous unless otherwise indicated, and mean an amount of a compound, peptide or composition of the present invention that is sufficient to improve the condition, disease, or disorder being treated and/or achieved the desired benefit or goal (e.g., control of body weight). Those skilled in the art will appreciate that the therapeutic effects need not be complete or curative, as long as some benefit is provided to the subject.

Determination of a therapeutically effective amount, as well as other factors related to effective administration of a compound of the present invention to a subject of this invention, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this invention is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

The above terms are relative to a reference or control. For example, in a method of enhancing glycosylation of α-DG in a subject of this invention by administering the controlled release composition to the subject, the enhancement is relative to the amount of glycosylation in a subject (e.g., a control subject) in the absence of administration of the controlled release composition.

The term “prevent,” “preventing” or “prevention of” (and grammatical variations thereof) refers to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

“Concurrently administering” or “concurrently administer” as used herein means that the two or more compounds or compositions are administered closely enough in time to produce a combined effect (that is, concurrently may be simultaneously, or it may be two or more events occurring within a short time period before and/or after each other, e.g., sequentially). Simultaneous concurrent administration may be carried out by mixing the compounds prior to administration, or by administering the compounds at the same point in time but at different anatomic sites and/or by using different routes of administration.

“Pharmaceutically acceptable” as used herein means that the compound or composition is suitable for administration to a subject to achieve the treatments described herein, without unduly deleterious side effects in light of the severity of the disease and necessity of the treatment.

### Pharmaceutical Formulations

The active compounds or agents described herein may be formulated for administration in a pharmaceutical carrier in accordance with known techniques. See, e.g., Remington, The Science and Practice of Pharmacy (21^{st} Ed. 2005). In the manufacture of a pharmaceutical formulation according to the invention, the active compound or agent is typically admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well-known techniques of pharmacy comprising admixing the components, and optionally including one or more accessory ingredients.

Furthermore, a “pharmaceutically acceptable” component such as a sugar, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as saline solutions, water, emulsions such as oil/water emulsion, microemulsions and various types of wetting agents.

Formulations suitable for oral administration may be presented in discrete units, such as capsules, cachets, lozenges, or tablets, each containing a predetermined amount of the active compound to achieve controlled rate of release and effective stable serum levels; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Further, the controlled-release composition of the present invention can be provided with liposomal formulations as are known in the art.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit/dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis/tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

In some embodiments of this invention, the compound of this invention is present in an aqueous solution for subcutaneous administration. In some embodiments, the compound is provided as a lyophilized powder that is reconstituted and administered subcutaneously.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: Ribitol Restores Functionally Glycosylated α-Dystroglycan and Improves Muscle Functions in FKRP Dystroglycanopathy

In this study, we tested our hypothesis in the FKRP mutant mice containing P448L mutation which is associated with CMD in clinic. Our results show that ribitol treatment increases levels of ribitol-5P and CDP-ribitol in muscle tissue and can effectively restore therapeutic levels of F-α-DG both before and after the onset of the disease phenotype. This results in significant improvement in muscle pathology and functions. Moreover, no side effects were detected in histology and functions of liver and kidney, muscle development, body weight and behavior of the animals. To the best of our knowledge, this is the first demonstration that a pentose alcohol ribitol constitutes a potentially effective and safe treatment to FKRP dystroglycanopathies.

One Month Treatment with Ribitol in Drinking Water Increases Glycosylation of α-DG in Cardiac and Skeletal Muscles.

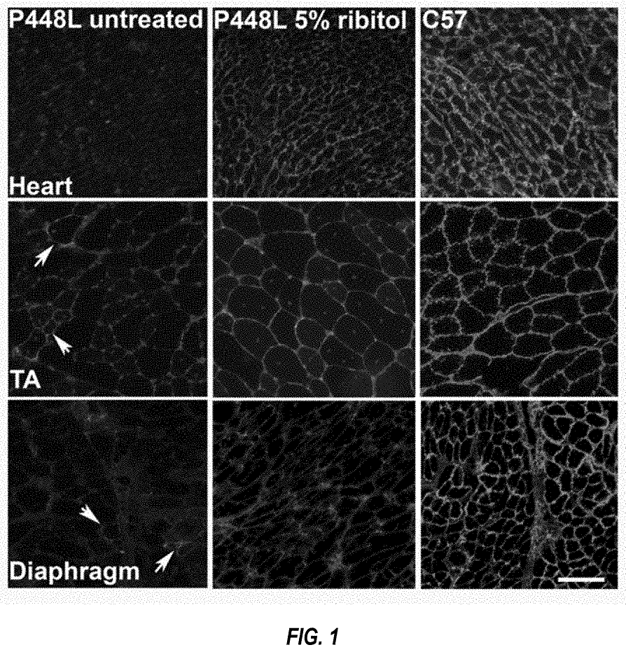

We have previously reported a FKRP mouse model containing a P448L mutation (P448L) with onset of the dystrophic pathology as early as 3 weeks of age. In the pilot experiment, 4-week-old P448L mice were treated with drinking water supplemented with 5% ribitol for 1 month. Glycosylation of α-DG was analyzed by immunohistochemistry with a monoclonal antibody, IIH6C4, specifically recognizing the laminin-binding epitopes of F-α-DG. Consistent with early reports, F-α-DG was undetectable in cardiac and skeletal muscles of the untreated P448L mice given drinking water only, except for isolated small clusters of revertant fibers in skeletal muscles, and one or two fibers expressing F-α-DG in cardiac muscle. (FIG. 1). In contrast, oral 5% ribitol treatment visibly increased F-α-DG in the heart, diaphragm and limb muscles. The signals of F-α-DG were consistently and clearly detected in the large proportion of diaphragm muscle fibers of the ribitol-treated mice. Interestingly, the signals for F-α-DG were easily detected with higher homogeneity in the cardiac muscle than in the skeletal muscles. Signals for F-α-DG in all the muscles of ribitol-treated mice were in general weaker when compared to the same muscle of C57 mice.

Oral Administration of Ribitol in Drinking Water Increases Levels of Ribitol-5P and CDP-Ribitol in Muscle Tissues.

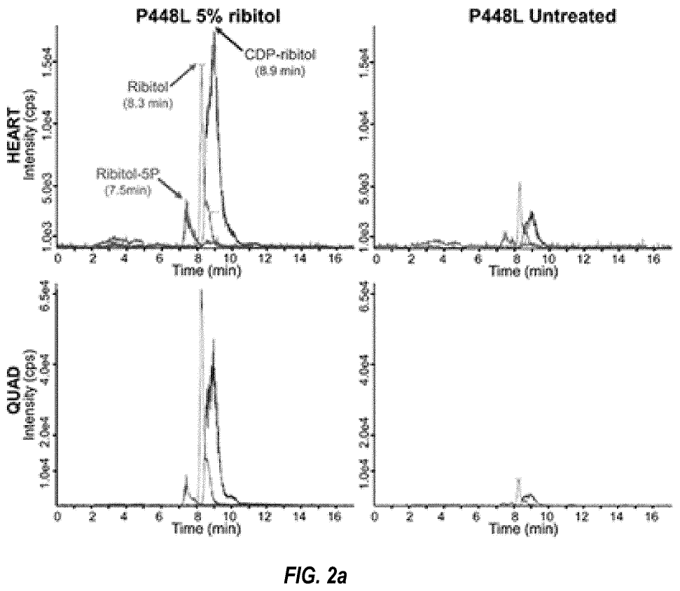

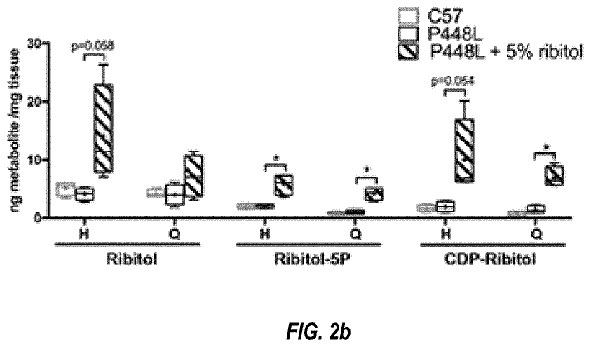

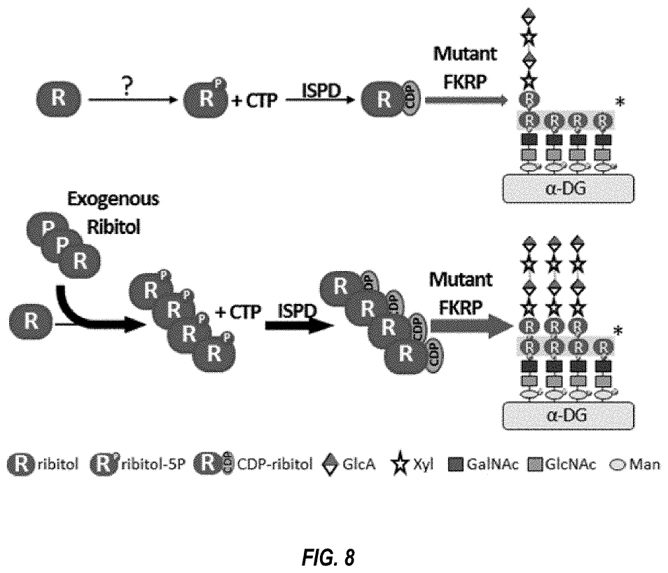

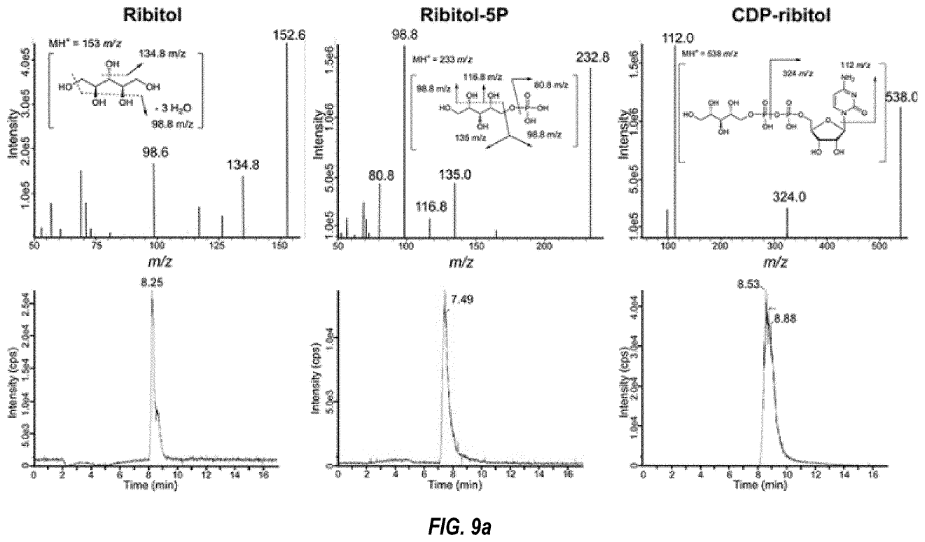

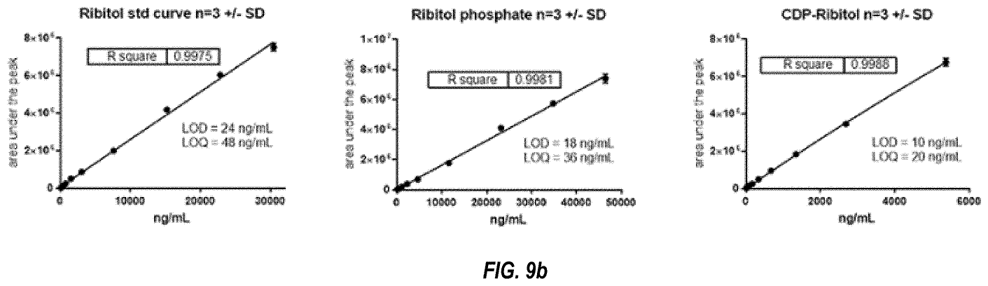

To evaluate whether oral administration of ribitol increases levels of ribitol-5P and CDP-ribitol in cardiac and skeletal muscles of mutant mice, we analyzed and quantified ribitol, ribitol-5P and CDP-ribitol in muscle tissues by LC/MS-MS. Ribitol (Sigma) as well as synthesized ribitol-5P and CDP-ribitol (Z-Biotech) were used to develop the detection method and to establish the standard curves for the quantification of the metabolites (FIGS. 9a and 9b, respectively). Endogenous levels of ribitol, ribitol-5P and CDP-ribitol were similar between untreated mutant P448L and C57 control mice (FIG. 2b). The three metabolites showed increased levels in heart and quadricep of the 5% ribitol-treated mice compared to untreated P448L mice (FIG. 2a and FIG. 2b). Levels of CDP-ribitol were at least 4-fold higher in heart and quadriceps of treated mice when compared to untreated and the difference of ribitol-5P and CDP-ribitol levels were statistically significant in both heart and quadricep (FIG. 2b). The levels of the metabolites were apparently higher in the heart tissues than in the skeletal muscles.

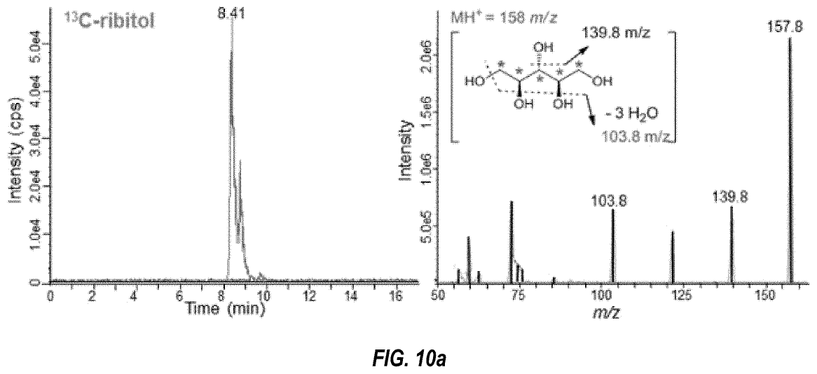

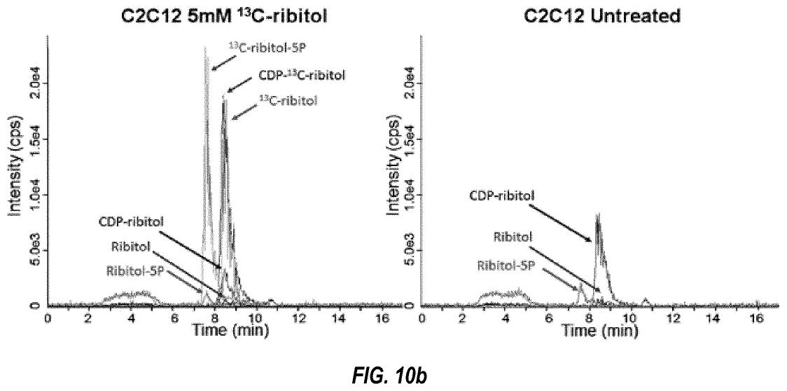

To address the question whether the orally administrated ribitol is, in fact, converted to ribitol-5P and CDP-ribitol, we treated differentiated C2C12 myotubes with isotopically labeled ¹³C5-ribitol in vitro. ¹³C5-ribitol (Omicron Biochemicals, Inc.) was used to develop the LC/MS-MS method for detection of ¹³C-ribitol in cell samples (FIG. 10a). The MRM (multi-reaction monitoring) methods for ¹³C-ribitol-5P and CDP-¹³C-ribitol were inferred from their non-labeled analogs (mass+5 amu). The LC/MS-MS analysis from the untreated cells showed low levels of endogenous ribitol, ribitol-5P and CDP-ribitol and absence of ¹³C-labeled analogs. However, the cells treated with ¹³C-ribitol showed clearly elevated levels of ¹³C-ribitol-5P and CDP-¹³C-ribitol as well as ¹³C-ribitol, but only background levels of endogenous analogs (ribitol, ribitol-5P and CDP-ribitol) as detected in the untreated cells (FIG. 10b). All together, these results confirm that exogenous ribitol can be converted to ribitol-5P and most importantly CDP-ribitol, the FKRP substrate for F-α-DG synthesis.

Long-Term Induction of Functionally Glycosylated α-DG by Ribitol in Severely Affected Mutant Mice.

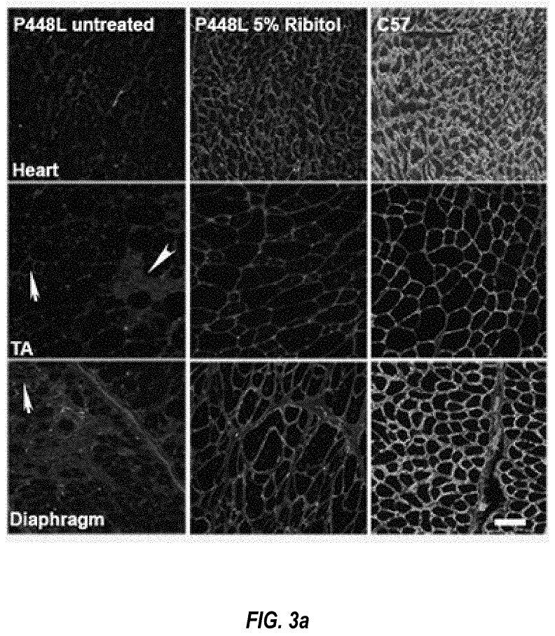

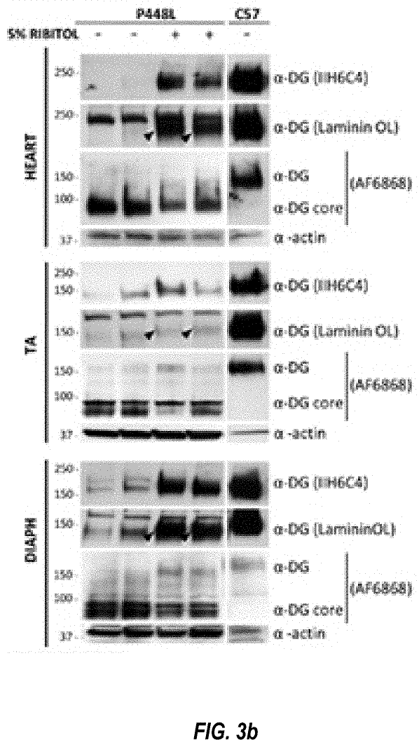

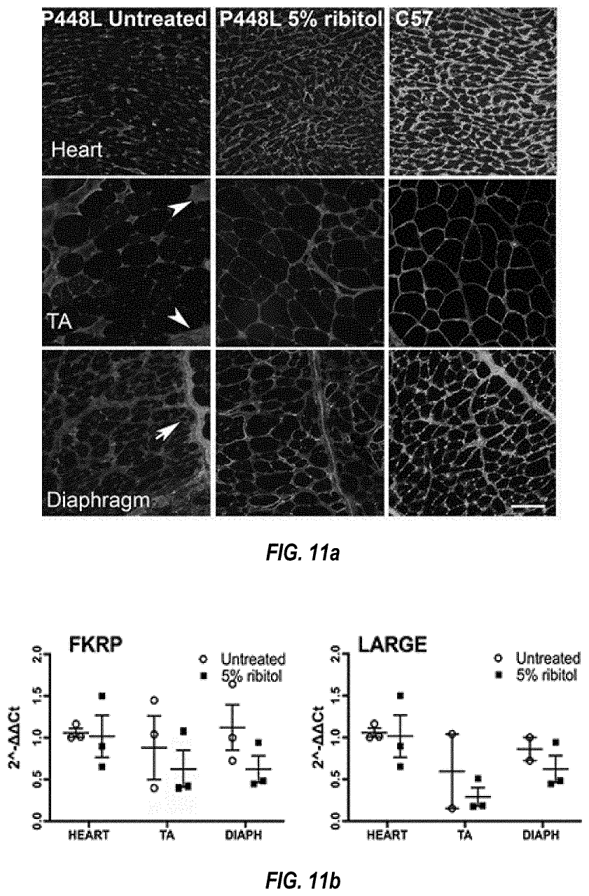

To assess whether ribitol treatment can maintain a long-term effect on glycosylation of α-DG in mutant mice already exhibiting severe dystrophic phenotype, we treated the P448L mice at the age of 7 weeks with 5% ribitol in drinking water for up to 3 and 6 months. Consistent with the 1 month treatment, all muscles from both cohorts of treated mice showed a clear increase in the levels of F-α-DG by immunofluorescence with IIH6C4 (FIG. 3a and FIG. 11a for 6 months and 3 months-treatments, respectively). Nearly all fibers in the cardiac muscle, and a majority of fibers in both diaphragm and limb muscles, were positive for F-α-DG (FIG. 3a). Signal distribution and intensity for F-α-DG were generally similar in the same muscles between 3 and 6 month ribitol-treated cohorts. The enhanced expression of F-α-DG by ribitol was further confirmed by western blot analysis with IIH6C4 antibody, reaching up to 14 and 17% of normal levels in the cardiac muscle and diaphragm, respectively (FIG. 3b and FIG. 3c). Enhanced expression of F-α-DG was further demonstrated by western blot with the antibody AF6868 (FIG. 3b). Finally, functionality of the ribitol-induced glycosylated α-DG was supported by laminin overlay assay (FIG. 3b).

To evaluate whether administration of ribitol affects expression of glycosyltransferases responsible for the synthesis of Core M3 glycan on alpha-dystroglycan, we measured levels of mutant FKRP and LARGE transcripts by quantitative real-time PCR in cardiac muscle, limb muscle and diaphragm (FIG. 11b). No statistically significant difference in FKRP and LARGE transcript levels was observed between treated and untreated samples in any of the tissues, suggesting that the effect of ribitol on levels of F-α-DG is independent to expression levels of the glycosyltransferases.

5% Ribitol Treatment in Drinking Water Alleviates Dystrophic Pathology in P448L Mice and Improves Respiratory Function.

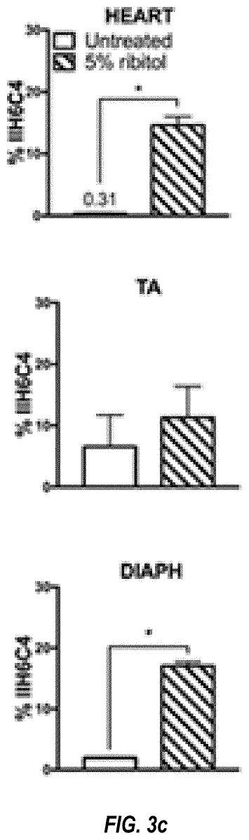

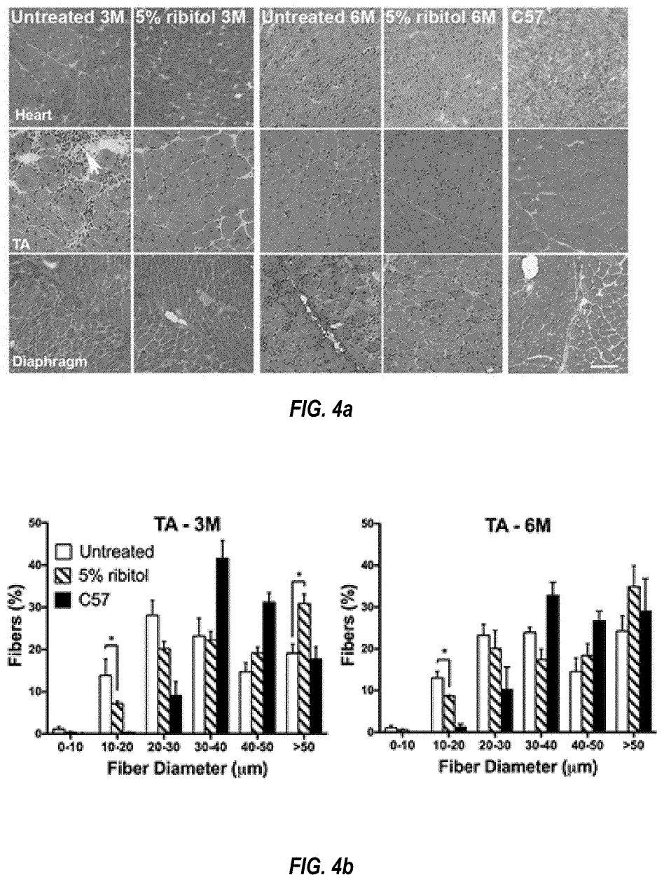

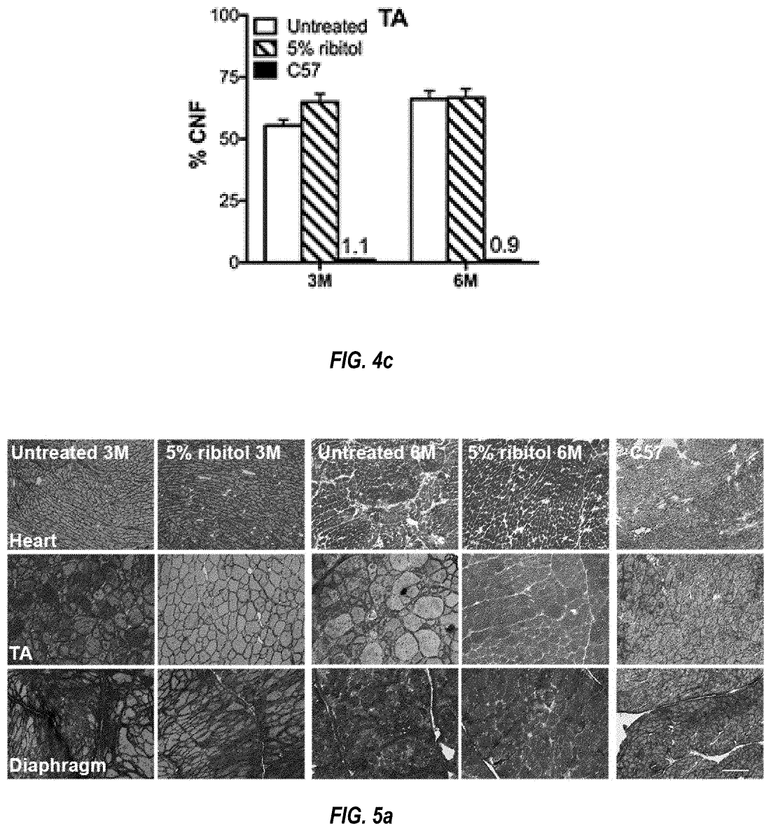

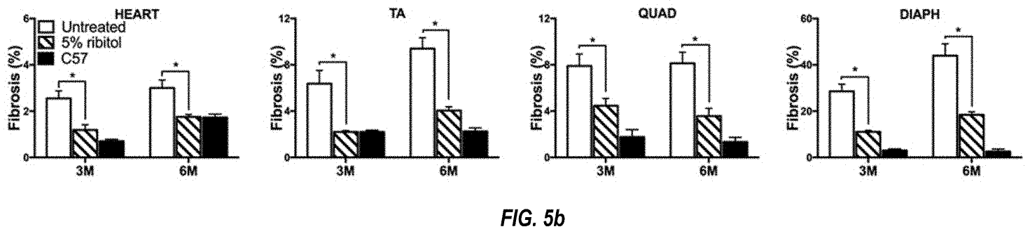

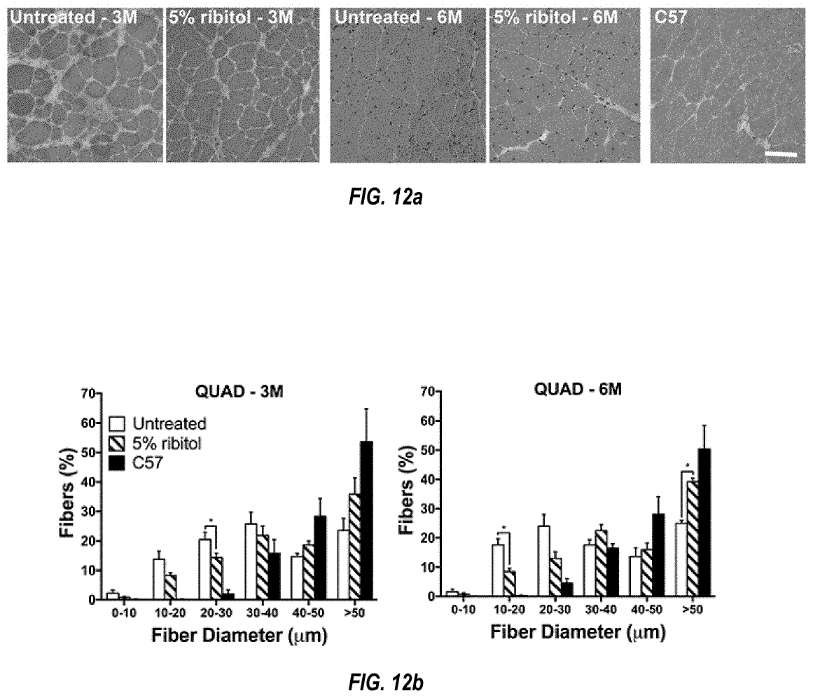

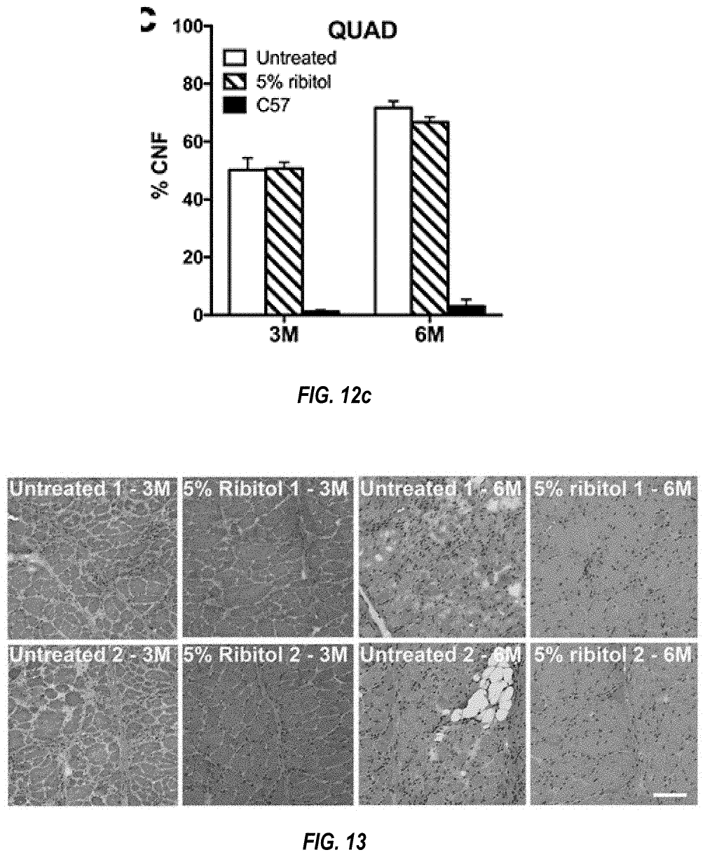

Therapeutic effect of 3 and 6 month treatments with 5% ribitol on dystrophic pathology of skeletal muscles was demonstrated by histology. Hematoxylin and eosin (H&E) staining showed the large areas of degenerating fibers, high variation in fiber sizes and high percentage of centrally nucleated fibers (CNF) in the skeletal muscles of the untreated P448L mice (FIG. 4a, FIG. 12a and FIG. 13). This was associated with focal inflammatory infiltrates. Treatment with ribitol improved the dystrophic pathology of limb muscles as evidenced by the diminished foci of necrotic fibers and a more homogenously distributed fiber size. Quantitative analysis from TA and quadriceps showed a statistically significant decrease in the number of fibers with small diameters (newly regenerated) indicating a decrease in degeneration after both 3 and 6-month ribitol treatments (FIG. 4b and FIG. 12b for TA and quadriceps, respectively). Furthermore, both 3 and 6 month ribitol treatments significantly decreased areas of fibrotic tissue detected by Masson's Trichrome staining when compared to untreated mice (FIG. 5a and FIG. 5b). No significant difference in percentage of CNF was observed between ribitol-treated and untreated P448L mice (FIG. 4c and FIG. 12c for TA and quadriceps, respectively). This is expected as significant CNF reduction could only be achieved with high dosage of viral particles with AAV gene therapy in the same mouse model.

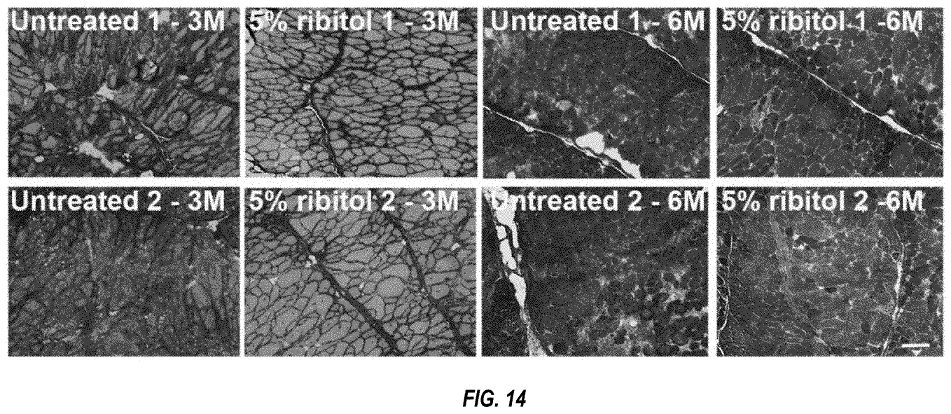

Importantly, 5% ribitol treatment significantly reduced pathology of the diaphragm. Large foci of degenerating fibers were common in the untreated diaphragms but became rarely observed in all the mice after 3 and 6 month ribitol treatments (FIG. 4a and FIG. 13). The most striking improvement was the degree of fibrosis. The diaphragm of the untreated mice showed heavy fibrosis at the 3 month time point (28.6% of tissue cross-section area), reaching more than 40% 6 months after the study initiation (FIG. 5a, FIG. 5b, and FIG. 14). However, the amount of fibrotic tissues in the ribitol-treated cohorts was significantly reduced to 11% and 18% after 3 and 6 month treatment, respectively.

The cardiac muscle of the P448L mice has limited pathology with only a small increase in fibrotic area as disease progresses. H&E staining did not show infiltration and degenerating fibers in both the ribitol-treated and the untreated mice (FIG. 4a). However, a significant reduction in fibrotic area was observed in the cardiac muscle of both 3 and 6 month ribitol-treated groups when compared to the untreated (FIG. 5a and FIG. 5b).

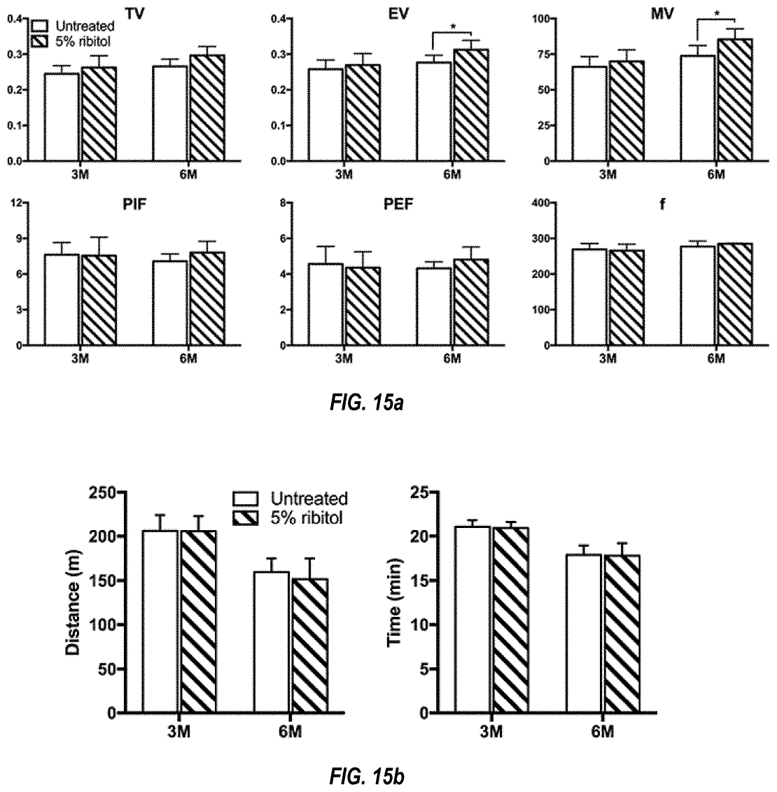

The significant improvement in histology of diaphragm with ribitol treatment was associated with improvement in respiratory function shown by whole-body plethysmography at 3-months and 6-months post-initiation of the treatment. A trend of improvement was observed in tidal volume (TV), expiratory volume (EV) and minute volume (MV) in both 3 and 6 month 5% ribitol-treated groups compared to the untreated P448L mice (FIG. 15a). Importantly, the improvement in EV and MV after 6 month ribitol treatment became statistically significant. Improvement was also observed in peak inspiratory flow (PIF) and peak expiratory flow (PEF) in the 6 month ribitol-treated group. However, significant improvement in limb muscle function was not demonstrated (FIG. 15b). Overall, these results showed that 5% ribitol oral treatment is able to enhance expression of F-α-DG with significant improvement in pathology of all muscles and in respiratory function.

Early Treatment with 10% Ribitol Significantly Improves Skeletal Muscle Function.

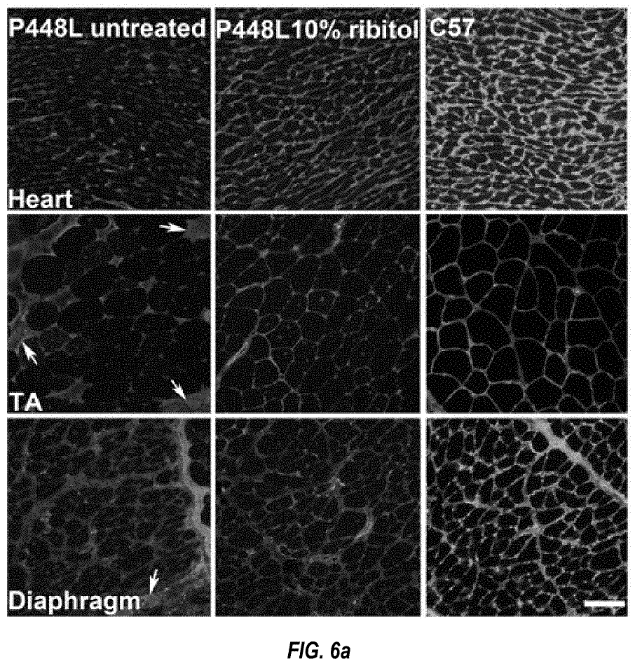

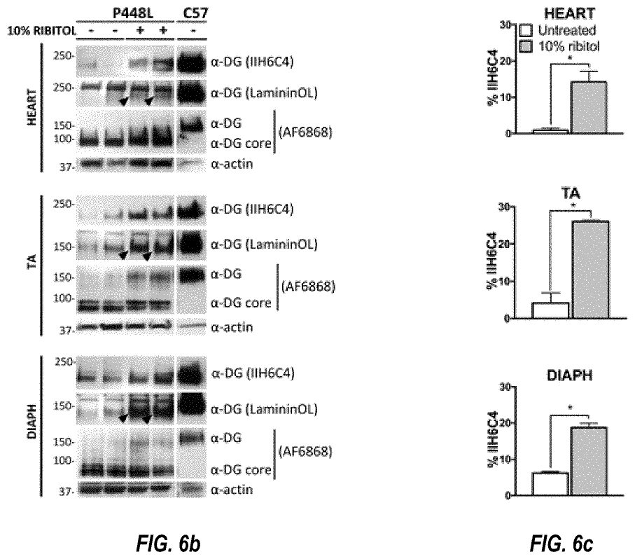

We reported recently that therapeutic outcome through restoration of F-α-DG depends on earlier treatment in the P448L mice. Specifically, significant improvement in skeletal muscle functions by AAV gene therapy is achieved before the onset of the disease, but not in adult mutant mice when disease phenotype is already well established. To assess whether early intervention with a higher dose of ribitol can achieve significant improvement in skeletal muscle functions, we initiated a treatment with 10% ribitol in drinking water to the breeding females when they became pregnant, and continued the treatment to the pups until they reached 19 weeks of age. All the functional tests were performed at the same age as the cohort treated with 5% ribitol from 7-week old for 3 months and age-matched untreated controls. Expression of F-α-DG was detected in all skeletal muscles and in the cardiac muscle of the 10% ribitol-treated mice by immunohistochemistry (FIG. 6a). F-α-DG was highly homogeneous in the cardiac muscle. Importantly, F-α-DG was clearly detected with even distribution in the skeletal muscles including the diaphragm. Expression of F-α-DG was clearly detected by western blots with the IIH6C4, reaching 14%, 18% and 26% normal levels in the heart, diaphragm and limb muscle respectively (FIG. 6b and FIG. 6c). Enhanced expression of F-α-DG was also demonstrated by western blot with the antibody AF6868 (FIG. 6b). Finally, functionality of the ribitol-induced glycosylated α-DG was supported by laminin overlay assay (FIG. 6b).

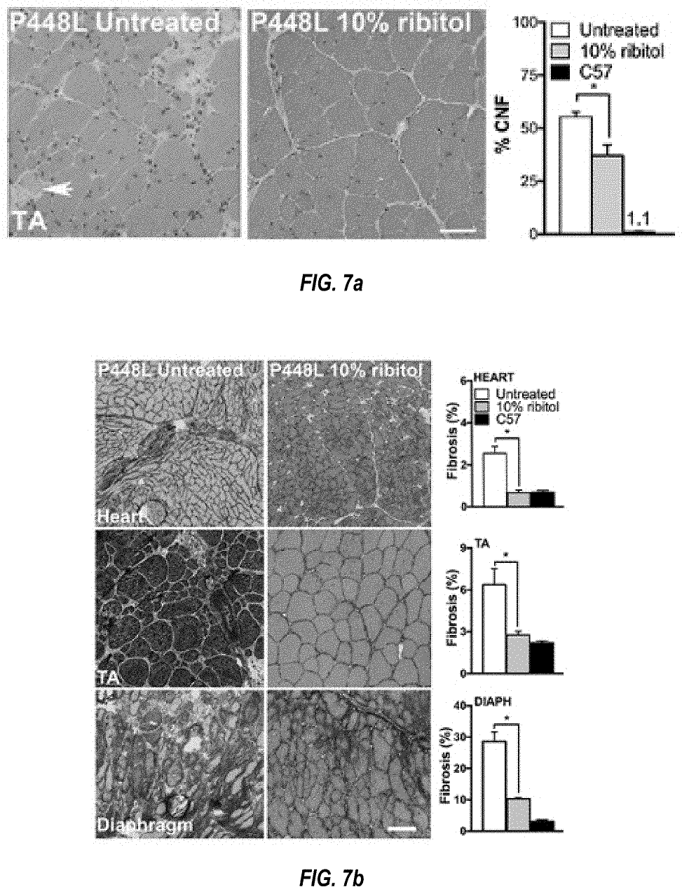

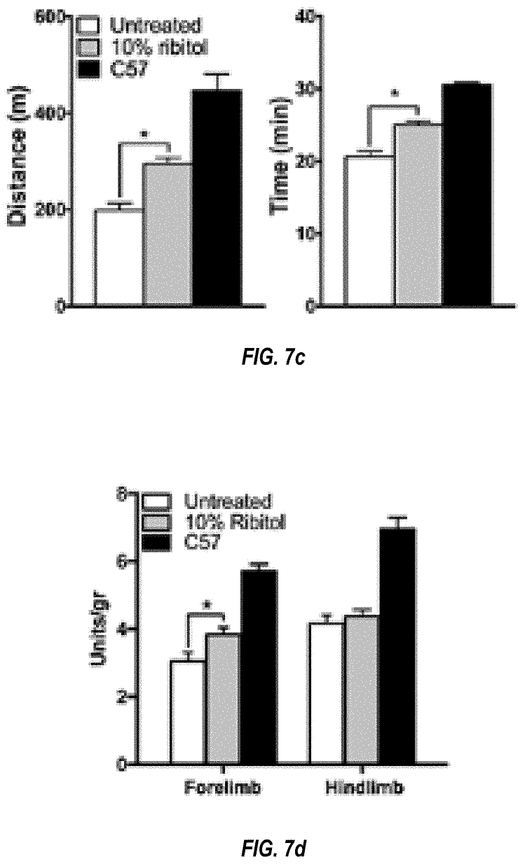

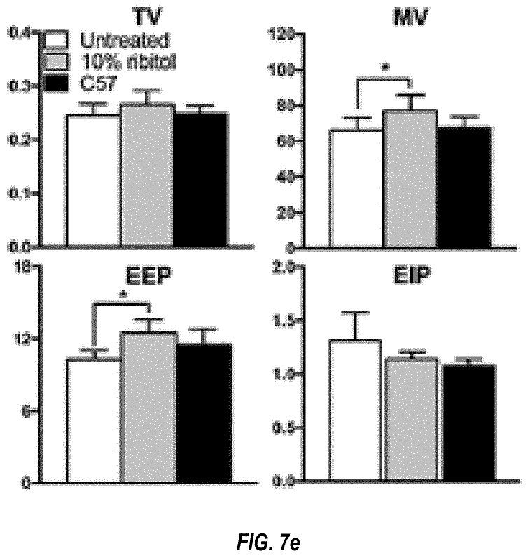

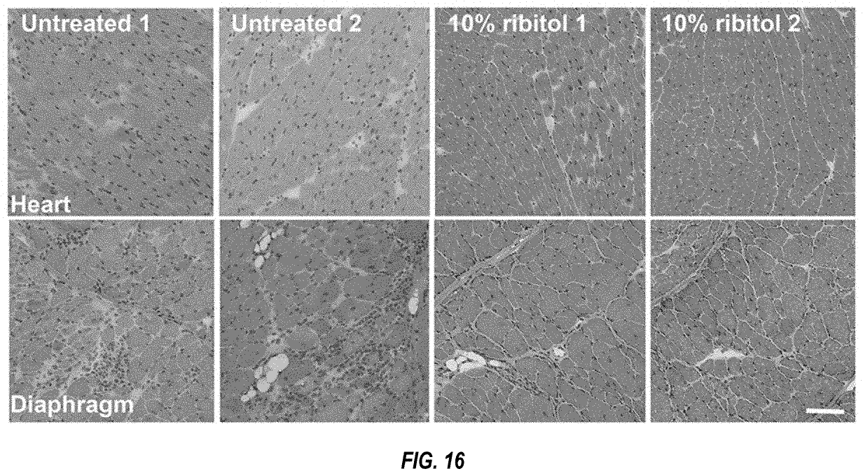

Consistent with the enhancement on the biochemical marker, dystrophic pathology in the 10% ribitol-treated mice was greatly alleviated with significantly fewer CNFs (FIG. 7a). Most fibers of the limb muscles were highly homogenous in shape and size and only a proportion of fibers were centrally nucleated within the diseased muscles. Notably, improvement in pathology with reduced infiltration and fiber size variation was also observed in the diaphragm (FIG. 16). Furthermore, reduction in fibrosis was significant in cardiac muscle, and most prominent in the diaphragm (FIG. 7b).

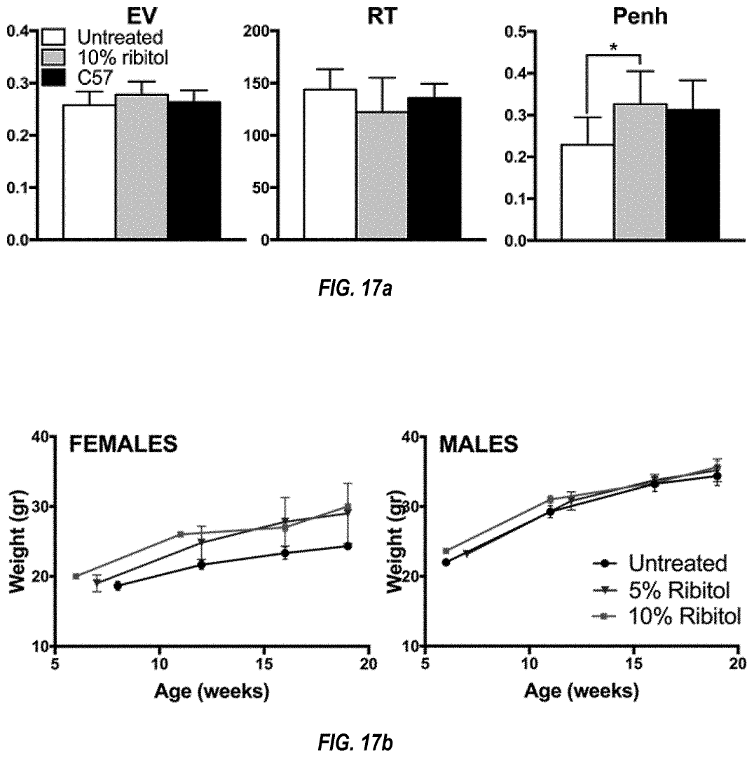

Importantly, early treatment with 10% ribitol significantly improved skeletal muscle functions of the P448L mice. Treadmill tests showed that both running distance and time of the treated mice were significantly longer than the age-matched untreated mice (FIG. 7c). Grip strength tests also showed significant improvement on forelimb force from the ribitol-treated mice compared to the untreated (FIG. 7d). Significant improvement in respiratory functions was also demonstrated by plethysmography (FIG. 7e and FIG. 17a). Similar to the mutant mice treated for 6 months with 5% ribitol, a trend of improvement in tidal yolume (TV), minute volume (MV), end-expiratory and end-inspiratory pause (EEP and EIP, respectively) was observed, with MV and EEP reaching significant difference between the 10% ribitol treated and the untreated P448L mice (FIG. 7e). Furthermore, improvement on expired volume (EV), relaxation time (RT) and enhanced pause (Penh) was also observed (FIG. 17a).

Effects of Ribitol Treatment on Body Weight and Histology of Liver, Kidney and Spleen.

No significant difference in body weight was observed between the mice treated with 10% ribitol from the embryonic stage, those treated with 5% ribitol from 7 weeks of age, or the age-matched untreated P448L mice at all time points although treated female mice were slightly heavier than the controls (FIG. 17b).

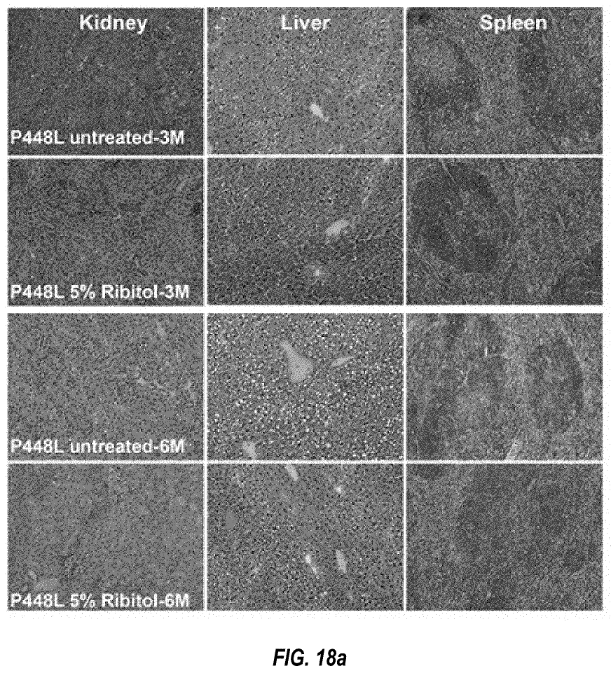

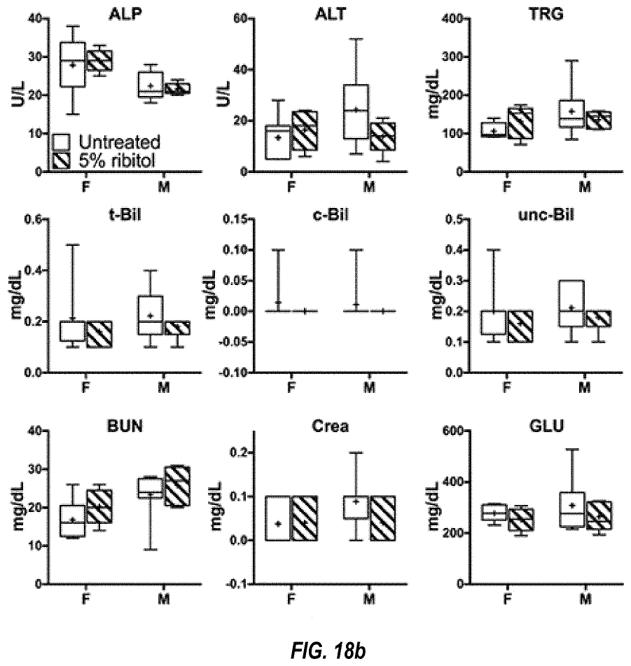

The effect of ribitol treatment the pharmacological concentration of 5% on histology of liver, kidney and spleen was also evaluated with H&E staining. All tissues showed normal structure without degeneration and inflammation, and no difference was observed between the untreated and the ribitol treated cohorts as illustrated in the FIG. 18a. We also performed biochemical analyses of serum markers for liver function including alkaline phosphatase (ALP), alanine transaminase (ALT), total bilirubin (t-Bil), conjugated bilirubin (c-Bil), and unconjugated bilirubin (unc-Bil). In addition, kidney function was evaluated by analyses of urea (BUN) and creatinine (Crea) levels. No statistical significance was observed between untreated and treated P448L mice. Levels of triglycerides (TRG) and glucose (GLU) were also similar between the two cohorts (FIG. 18b).

Despite significant advances in understanding the causes and clinical manifestation of dystroglycanopathies, almost no progress has been made for the treatment of the diseases including those caused by FKRP mutations. Currently, physical therapy and other clinic management routinely provided to patients only serve as palliative care. The only option of pharmacological intervention available is glucocorticoid steroids which are being used anecdotally based on reported benefits from other muscular dystrophies, especially Duchenne muscular dystrophy (DMD). Experimental therapy with the aim to restore F-α-DG by AAV-mediated gene therapy has been reported with high efficacy in preventing disease progression in mouse models. However, clinical trials of the therapy for the diseases with such a wide range of phenotypes are challenging and remain to be conducted. Therefore, there is an urgent need for developing experimental therapies. Here we show that ribitol, a natural pentose alcohol present in some plants and animals and considered as a metabolic intermediate or end-product, can effectively restore therapeutic levels of F-α-DG and, more importantly, ameliorate dystroglycanopathy caused by the FKRP P448L mutation which is associated with severe CMD phenotype in clinic. Our results offer a potentially safe and effective new class of treatment for restoration of F-α-DG to FKRP dystroglycanopathies. This treatment could be applied in combination with other therapies, such as AAV gene therapy for higher efficacy by enhancing the function of FKRP transgene. The results also raise the potential of developing similar approaches for enhancing F-α-DG in cells of other diseases associated with aberrant O-mannosylation of α-DG. An example of such application is for cancers exhibiting reduced or lack of F-α-DG in association with invasion and metastasis which can be inhibited by gene transfer-mediated upregulation of F-α-DG.

FKRP dystroglycanopathy affects respiratory and cardiac muscles even in diseases with mild defects in skeletal muscles. Failures in respiratory and cardiac functions are the prime causes for the lethality of the diseases. Therefore, restoration of F-α-DG and improvement in cardiac and respiratory functions are critically important for life quality and longevity of patients. Ribitol treatment enhances F-α-DG in both cardiac and diaphragm muscles which is often most severely affected. This leads to significant improvements in the pathology of the diaphragm with striking reduction in fibrosis which may explain the enhancement of respiratory functions. Cardiac defects in both pathology and functions in the P448L mice are limited and significant improvement in function is difficult to demonstrate even with effective AAV9 gene therapy. Nevertheless, ribitol treatment is able to produce sustained and homogenous expression of F-α-DG in the treated cardiac muscle, resulting in significant reduction in fibrosis. All the data therefore clearly demonstrate therapeutic potential of the treatment to the two critical organs and their functions. Also important, ribitol treatment of different time frames up to 6 months shows no clear side effect. Oral ribitol administration from pregnancy to adult of the P448L mice does not affect pregnancy, embryo development, body weight and overall behavior of the mutant mice. These together with normal histology and levels of serum markers for liver and kidney suggest the potential in safety for clinic applications.

Therapeutic effects of ribitol treatment are related to the enhanced F-α-DG. Ribitol supplementation has been shown to increase the levels of CDP-ribitol both in cultured cells and in muscles of wild-type mice in vivo. Our detection of increased levels of CDP-ribitol in both cardiac and skeletal muscles of FKRP mutant mice is consistent with the earlier report. We also demonstrated the increase in the levels of ribitol-5P and CDP-ribitol which is considered the substrate of ISPD, indicating that ribitol can be effectively converted to CDP-ribitol in both FKRP mutant muscle tissues as well as normal tissues. FKRP function is considered essential for functional glycosylation of α-DG. It is intelligible that the enhanced expression of F-α-DG in the diseased muscles also requires function of FKRP as in normal muscles. Indeed, it has long been demonstrated that diseased muscles with missense mutations of the FKRP gene remain capable of producing F-α-DG but at variable lower levels (hypoglycosylation). This is most convincingly demonstrated in mouse models with FKRP mutations. The mutant mice with common L276I mutations express low but clearly detectable levels of F-α-DG in both skeletal and cardiac muscles. Considerable amount of F-α-DG is also detected in all compound heterozygotes containing L2 761 allele.

Despite the lack of expression of F-α-DG in the majority of muscle fibers of the P448L mice, individual muscle fibers are able to express near normal levels of F-α-DG with laminin-binding capacity. Normal levels of F-α-DG are expressed in all regenerating fibers. More direct evidence came from AAV-mediated expression of the P448L mutant FKRP, which is able to restore F-α-DG and protect muscles from degeneration. Interestingly, strong expression of F-α-DG is detected in all new born skeletal and cardiac muscles of the P448L mice and this expression is not associated with clear increase in mutant FKRP expression. This demonstration supports the functionality of the mutant FKRPs and more importantly suggests that factor(s) other than FKRP expression could compensate for the reduced functionality of the mutant FKRPs.

We therefore hypothesize that the additional amount of ribitol allows the muscle fibers to produce higher than normal levels of FKRP substrate (CDP-ribitol) which enhances and partially compensates for the reduced function of mutant FKRPs (FIG. 8). Fortunately, more than 90% of patients with FKRP mutations contain at least one allele of the L276I mutation which retains at least partial function as demonstrated in patient muscles and in the mutant mouse models. Therefore, this low cost and easy to administer experimental therapy will likely be applicable to the majority of patients with FRKP mutations. This, together with the nature of the drug, expected to be of limited side effects, would make execution of an early clinical trial much simpler. Further studies to understand the pharmacokinetics of ribitol in disease models and metabolic pathways of the pentose alcohol to CDP-ribitol would also allow us to optimize the dosage and delivery regime for higher efficacy. Since details of the mammalian ribitol-5P biosynthetic pathway remains unknown, potential non-glycosylation-related effect of pharmacological dose of ribitol on muscles and other systems requires attention.

Animal Care.

All animal studies were approved by the Institutional Animal Care and Use Committee (IACUC) of Carolinas Medical Center. All mice were housed in the vivarium of Carolinas Medical Center following animal care guidelines of the institute. Animals were ear tagged prior to group assignment. Food and water were available ad libitum during all phase of the study. Body weight was measured from 6 weeks to 19 weeks of age.

Mouse Model and Experimental Procedure.

FKRP P448L mutant mice were generated by the McColl-Lockwood Laboratory for Muscular Dystrophy Research. The mice contain a homozygous missense mutation (c.1343C>T, p.Pro448Leu) in the FKRP gene with the floxed neomycin resistant (Neo^{r}) cassette removed from the insertion site. C57BL/6 (wild-type/C57) mice were purchased from Jackson Laboratory.

Ribitol was purchased from Sigma (A5502 Adonitol, ≥98%, Sigma, St. Louis) and dissolved in drinking water to the final concentration of 5% or 10%. P448L mice aged at 4 weeks were treated with 5% ribitol drinking water for 1 month and P448L mice aged at 7 weeks were treated with 5% ribitol drinking water for 3 months and 6 months. All the mice were randomly assigned to either treatment or control groups. And a minimum number of 4 mice were used for each group. No animal was excluded. P448L female breeders were given 10% ribitol in drinking water during pregnancy, and pups continued to be treated with 10% ribitol in drinking water until they were euthanized at 19 weeks of age. Untreated age-matched P448L and wild-type C57BL/6 mice were used as controls. The animals were terminated at the end of each treatment time point and tissues including heart, diaphragm, TA, quadriceps, liver, spleen and kidney were collected for analyses.

Immunohistochemical and Western Blot Analysis.

Tissues were dissected and snap-frozen in dry-ice-chilled-2-methylbutane. For immunohistochemical detection of functionally glycosylated α-DG, 6 m of thickness cross sections of untreated and C57 control, as well as tissues from treated cohorts were included in each slide. Slides were first fixed in ice cold Ethanol:Acetic acid (1:1) for 1 min, blocked with 10% normal goat serum (NGS) in IxTris-buffer saline (TBS) for 30 min at room temperature, and incubated overnight at 4° C. with primary mouse monoclonal antibody IIH6C4 (EMD Millipore) (1:500) against α-DG. Negative controls received 10% normal goat serum in 1×TBS only. Sections were washed and incubated with secondary AlexaFLuor 488 goat anti-mouse IgM (Invitrogen) (1:500) at room temperature for 2 hr. Sections were washed and finally mounted with fluorescence mounting medium (Dako) containing 1×DAPI (4′,6′-diamidino-2-phenylindole) for nuclear staining. Immunofluorescence was visualized using an Olympus BX51/BX52 fluorescence microscope (Opelco) and images were captured using the Olympus DP70 digital camera system (Opelco). Slides were examined in a blinded manner by the investigator.

For western blot analysis, tissues were homogenized in extraction buffer (50 mM Tris-HCl pH 8.0, 150 mM NaCl, and 1% Triton X-100), supplemented with 1× protease inhibitor cocktail (Sigma-Aldrich). Protein concentration was quantified by Bradford assay (Bio-Rad DC protein assay). Eighty μg of protein was loaded on a 4-15% Bio-Rad Mini-PROTEAN TGX gel (Bio-Rad) and immunoblotted. Amount of total protein loaded for C57 mice was half of the amount loaded for the P448L mice. Nitrocellulose membranes (Bio-Rad) were blocked with 5% milk in 1×PBS for 2 hr at room temperature and then incubated with the following primary antibodies overnight at 4° C.: IIH6C4 (1:2000), AF6868 (R&D Systems) (1:1000) and α-actin (Sigma) (1:1000). Appropriate horseradish peroxidase (HRP)-conjugated secondary antibodies were incubated for 2 hr at room temperature. All blots were developed by electrochemiluminescence immunodetection (PerkinElmer). For IIH6C4 band quantification from western blot ImageJ software was used. For laminin overlay assay, nitrocellulose membranes were blocked with laminin overlay buffer (10 mM ethanolamine, 140 mM NaCl, 1 mM MgCl2, and 1 mM CaCl₂, pH 7.4) containing 5% nonfat dry milk for 1 hr at 4° C. followed by incubation with laminin from Engelbreth-Holm-Swarm murine sarcoma basement membrane (L2020) (Sigma) at a concentration of 2 μg/ml overnight at 4° C. in laminin overlay buffer. Membranes were then incubated with rabbit anti-laminin antibody (Sigma) (1:1500) followed by goat anti-rabbit HRP-conjugated IgG secondary antibody (Santa Cruz Biotechnology) (1:3000). Blots were saturated with Western-Lightning Plus ECL (Perkin-Elmer) before exposure to and developing of GeneMate auto-radiographic film (VWR).

Histopathological and Morphometric Analysis.

Frozen tissues were processed for hematoxylin and eosin (H&E) and Masson's Trichrome staining following standard procedures. Muscle cross-sectional fiber equivalent diameter was determined from tibialis anterior and quadriceps stained with H&E using MetaMorph v7.7 Software (Molecular Devices). Percentage of centrally nucleated myofibers were manually quantified from the same tissue sections stained with H&E. Fibrotic area represented by blue staining in the Masson's Trichrome stained sections was quantified from heart, diaphragm, tibialis anterior and quadriceps using ImageJ software. For all the morphometric analyses, a total of 300 to 400 fibers from two representative 20× magnification images per each muscle per animal were used.

Quantitative Reverse Transcriptase PCR Assay.

Tissues were collected from heart, diaphragm and tibialis anterior. RNA was extracted using TRIzol (Invitrogen) following the supplied protocol. Final RNA pellet was re-suspended in 20 μl RNAse-nuclease free water. Final RNA concentration was determined using Nanodrop 2000c. One μg of RNA was subsequently converted to cDNA using the High-Capacity RNA-to-cDNA™ Kit (Applied Biosystems) following the supplied protocol. cDNA was then used for quantitative real-time PCR using the mouse FKRP-FAM (Mm00557870_m1) and LARGE-FAM (Mm00521885 ml) taqman assay with primer limited GAPDH-VIC (Mm99999915_g1) as the internal control and TaqMan® Universal Master Mix II, with UNG (Life Technologies). Quantitative real time PCR was run on the BioRad CFX96 Touch™ Real-Time PCR Detection System (BioRad) following the standard real time PCR conditions suggested for taqman assays. Results of FKRP and LARGE transcript were calculated and expressed as 2\hat{ }-ΔΔCt and compared across tissues and animals.

Metabolite Extraction from Muscle Tissues and LC/MS-MS Analysis.

Ribitol was purchased from Sigma (A5502). Ribitol-5P and CDP-ribitol were synthesized by Z Biotech (Aurora, Colo.). Muscle tissues were collected, and blinded samples were subjected to the following procedure. Thirty to 80 μg of frozen tissue samples were homogenized with 400 μl of MeOH:Acetonitrile (ACN) (1:1) and then centrifugated for 5 min at 10,000 rpm. The supernatants were removed, transferred to individual wells of 96-well plate and analyzed by LC/MS-MS. An Applied Biosystems Sciex 4000 (Applied Biosystems; Foster City, Calif.) equipped with a Shimadzu HPLC (Shimadzu Scientific Instruments, Inc.: Columbia, Md.) and Leap auto-sampler (LEAP Technologies; Carrboro, N.C.) were used to detect ribitol, ribitol-5P and CDP-ribitol from tissue samples and synthetic compounds. The metabolites were separated on a silica gel column (Hypersil Silica 250×4.6 mm, 5 micron particle size) using solvent A: water, 10 mM NH₄OAc, 0.1% formic acid and solvent B: MeOH:ACN (1:1). The following gradient was used: 0-12 min, 5% buffer B; 13-14 min, 95% buffer B, 15-17 min, 5% buffer B. Under these conditions, ribitol, ribitol-5P and CDP-ribitol eluted at 8.3 min, 7.5 min and 8.9 min, respectively. The metabolites were analyzed using electrospray ionization mass spectrometry operated in positive ion mode, ESI+. Compounds concentration in tissue samples were determined based on standard curves prepared by serial dilutions (200-0.01 μM) of each of the compound in MeOH:ACN (1:1).

Cell Culture and LC/MS-MS Analysis of Isotopically Labeled Metabolites.

C2C12 mouse myoblast (ATCC, CRL-1772) were seeded and grown in DMEM GlutaMax medium (Gibco by Life Technologies) supplemented with 10% fetal bovine serum and 100 μg/ml penicillin-streptomycin. Differentiation into myotubes was induced by replacing the growth media with DMEM supplemented with 1 μM Insulin (Sigma), 2% heat-inactivated horse serum (Gibco by Life Technologies), 2.5 μM Dexamethasone (Sigma) and 5 mM ¹³C5-ribitol (Omicron Biochemicals, Inc., South Bend, Ind. USA) when cells reached confluence. Cells were harvested 5 days later and analyzed by LC/MS-MS for detection of ribitol (153.2-98.8 m/z), ribitol-5P. (233.1→98.8 m/z), CDP-ribitol (538.1→324.1 m/z), ¹³C-ribitol (158.3→103.8 m/z), ¹³C-ribitol-5P (238.1→98.8 m/z) and CDP-¹³C-ribitol (543.1.→324.1 m/z) as described above.

Muscle Function Tests.

For treadmill exhaustion test, 17 and 30 weeks old mice were placed on the belt of a five-lane-motorized treadmill (LE8700 treadmill, Panlab/Harvard Apparatus, Barcelona, Spain) supplied with shock grids mounted at the back of the treadmill, which delivered a 0.2 mA current to provide motivation for exercise. Initially, the mice were subjected to an acclimation period (time, 5 min; speed, 8 cm/s, and 0° incline). Immediately after acclimation period, the test commenced with speed increases of 2 cm/s every minute until exhaustion. The test was stopped and the time to exhaustion was determined when the mouse remained on the shock grid for 5 s without attempting to re-engage the treadmill. For grip force test, forelimb and hindlimb in peak torque was measured by a grip strength meter (Columbus Instruments). For forelimb force, the animal was held so that only the forelimb paws grasp the specially designed mouse flat mesh assembly, and was pulled back from the tail until the grip was broken. The force transducer recorded the peak force reached when the animal's grip is broken. For hindlimb force, an angled mesh assembly was used. Mice were allowed to rest on the angled mesh assembly, facing away from the meter with its hindlimbs at least one-half of the way down the length of the mesh. The mouse tail was pulled directly toward the meter and parallel to the mesh assembly. During this procedure, the mice resist by grasping the mesh with all four limbs. Pulling toward the meter was continued until the hindlimbs released from the mesh assembly. Five successful hindlimb and forelimb force measurements within 2 minutes were recorded. The average value was used for analysis. Force was presented as values of KGF (kilogram-force) units normalized to bodyweights (gr) as “Units/gr”. The tests were performed 1 week before euthanasia.

Whole Body Plethysmography.

Respiratory functional analysis in conscious, freely moving 18 and 31 weeks old mice were measured using a whole-body plethysmography technique. The plethysmograph apparatus (Emka Technologies, Falls Church, Va.) was connected to a ventilation pump for the purpose of maintaining a constant air flow, a differential pressure transducer, a usbAMP signal amplifier, and a computer running EMKA iox2 software with the respiratory flow analyzer module, which was used to detect pressure changes due to breathing and recording the transducer signal. An initial amount of 20 mL of air was injected and withdrawn via a 20 mL syringe into the chamber for the purpose of calibration. Mice were placed inside the “free moving” plethysmograph chamber and allowed to acclimate for 5 min in order to minimize any effects of stress related changes in ventilation. Resting ventilation was measured for a duration of 15 min after the acclimation period. Body temperatures of all mice were assumed to be 37° C. and to remain constant during the ventilation protocol.

Statistical Analysis.

All data are expressed as mean±SEM unless stated otherwise. Statistical analyses were performed with GraphPad Prism version 7.01 for Windows (GraphPad Software). Individual means were compared using multiple t tests. Differences were considered to be statistically significant at p≤0.05 (*).

### Example 2. Controlled Release of Ribitol/Ribose for Treating Muscle Weakness and Muscular Dystrophy

Muscle weakness is a common condition which can be caused by aging or muscle diseases such as muscular dystrophy. One important factor for maintaining muscle integrity and function is the effective connection between muscle fibers and non-fiber tissue within muscles. This connection makes muscle strong and prevents contraction-related damage. This connection is made up of several different molecular linkages, one of which is made through the binding of a sugar modified dystroglycan protein on muscle fiber membrane. Defects of the sugar modification of the protein are known to be caused by mutations (defects) of many genes including the gene fukutin-related protein (FKRP). Lack of this important sugar-mediated linkage causes muscle degeneration and loss of function. Eventually patients will lose mobility. Muscle damage can also affect the diaphragm and heart, leading to failure of respiratory and cardiac functions, and finally shorten the life. There is no effective treatment for FKRP defect related diseases. Recently, the structure of the sugar in the dystroglycan has been decoded and contains ribitol 5-phosphate within the sugar chain. This ribitol 5-phosphate is considered to be added to the sugar chain by the function of FKRP. Surprisingly, we recently found that supplement of ribitol and ribose can diminish disease pathology of the muscular dystrophy caused by FKRP mutations. While the mechanism(s) is not clearly understood, this approach provides a promising therapy to the devastating disease which has so far had no effective treatment.

Therapeutic effect has been achieved in an animal model of the disease via drinking water containing high doses of ribitol or ribose, e.g., from 5 g/kg and 10 kg/kg body weight daily. This amount of ribitol or ribose, if translated to human applications with 12.3 times reduction rate, recommended by the FDA as described in “Conversion of Animal Doses to Human Equivalent Doses Based on Body Surface Area,” is difficult to manage in the clinic for long term use as the human equivalent doses will be up to about 0.5 g to 1 g/kg body weight. This amounts to 20 g to 40 g active drug ingredient daily with a subject's body weight of 20 kgs to 40 kgs.

Such a large daily amount is not only difficult to manage, but also likely has health consequence as would be expected from the consumption of this amount of glucose daily. Furthermore, delivery via drinking water or any other form with unlimited times daily is difficult to apply in the clinic as a therapy for treating a chronic disease. This will also lead to high variation in serum levels, and thus variation in therapeutic effects. Alternatively, a higher daily dose can be delivered once, twice, 3 or 4 times a day. However, the metabolites are known to be cleared quickly from the serum (Clin Pharmacol. 2018; 10: 73-78), with a half-life of clearance of about 30 minutes, mostly through kidney. This together with uptake by bodywide tissues will lead to a very limited time period with therapeutic levels of serum ribitol/ribose. One option to compensate for this undesirable serum levels limit is to increase the dose further for achieving therapeutic effect, but this is not a good solution.

This invention provides a formulation for ribitol and ribose to be taken orally with controlled release to achieve stable serum levels with daily oral administration. To do so, we first established normal serum levels of ribitol and ribose (up to 162 ug/L (Table 1)) and 5 mg/L respectively (EBioMedicine 2017 November; 25:143-153). We then determined the levels of the drugs in the serum of subject animals treated with ribitol/ribose at the effective dose via drinking water (5 g/Kg and 10 g/Kg daily). Based on these data, we designed a formulation for stable release so that the drug can be taken once, twice or three times a day and serum levels of the drugs can be maintained constant at the desirable therapeutic levels. As a result, this controlled release will permit the use of a lower dosage offered by the favorable pharmacokinetics under this formulation. Consequently and also importantly, a lower dose can reduce any potential side effect especially related to the long-term use at high dose under non-formulated conditions.

This invention applies formulations to achieve constant release of the active drug ingredient ribitol and ribose from about 2 hours to about 24 hours by single oral administration to maintain therapeutic serum levels of the active ingredient, from 200 ug/L to 20 mg/L, preferably between 0.5 mg/L to 5 mg/L. The desirable constant serum levels of the drugs can be achieved, e.g., by micro-encapsulation, or osmotic controlled-release oral delivery systems (OROS) with a semi-permeable outer membrane and one or more small laser drilled holes in it. In the latter method, as the tablet passes through the body, water is absorbed through the semipermeable membrane via osmosis and the resulting osmotic pressure pushes drug out gradually and the rate can be controlled by the size of the hole and addition of excipient(s).

In some embodiments, a matrix of the ribitol and/or ribose with a polymer as gelling agent, which can be a cellulose derivative, non-cellulose natural, and/or polymers of acrylic acid, can be used. A nonlimiting example of a gelling material includes METHOCELDC2 (Hypromellose), optionally in combination with a portion of microcrystalline cellulose and/or the colloidal silicon dioxide. The mixture can be blended in a suitable mixer until homogeneously mixed. Magnesium stearate can be added and blended for a few minutes. The mixture can then be compressed to tablets, with a suitable film coat to impart mechanical strength, and/or encapsulated to enhance appearance and product stability and improve patient compliance. Many film coating systems are available, as are known in the art.

This invention discloses the use of the controlled release to greatly reduce the daily amount of active ingredient ribitol and ribose in some embodiments from about 0.5 g/kg-1 g/kg bodyweight via conventional oral delivery to about 0.05 g/kg-0.1 g/kg bodyweight (a 10 times reduction). This disclosure makes long-term administration practically possible and is expected to greatly reduce potential side effects.

This disclosure applies methods of formulation to control the release of ribitol and ribose in the gastrointestinal tract to achieve constant and desirable therapeutic serum levels of the drugs, from about 200 ug/L to about 20 mg/L, e.g., from about 0.5 mg/L to about 5 mg/L.

With this disclosure, practically feasible oral administration of a therapeutic amount of ribitol and ribose can be applied for treatment of muscle weakness.

With this disclosure, practically feasible oral administration of a therapeutic amount of ribitol and ribose can be applied for treatment of muscular dystrophy.

With this disclosure, practically feasible oral administration of a therapeutic amount of ribitol and ribose can be applied for treatment of FKRP-mutation-related muscular dystrophy.

In some embodiments, this invention can be applied to treat muscle weakness in combination with any other treatment, including but not limited to, a myostatin inhibitor, which increases muscle size.

In some embodiments, this invention can be applied to treat FKRP-mutation-related muscular dystrophy in combination with gene therapy, wherein the FKRP gene product is produced to compensate for the loss of FKRP function in individuals with FKRP-mutation-related muscular dystrophy.

Methods for Serum Ribitol Measurement by LC/MS-MS Analysis.

Ribitol was purchased from Sigma (A5502). Sera were collected, and blinded samples were placed in individual wells of a 96-well plate and analyzed by LC/MS-MS. An Applied Biosystems Sciex 4000 (Applied Biosystems; Foster City, Calif.) equipped with a Shimadzu HPLC (Shimadzu Scientific Instruments, Inc.: Columbia, Md.) and Leap auto-sampler (LEAP Technologies; Carrboro, N.C.) were used to detect ribitol from samples and synthetic compound. The metabolite was separated on a silica gel column (Hypersil Silica 250×4.6 mm, 5 micron particle size) using solvent A: water, 10 mM NH₄OAc, 0.1% formic acid and solvent B: MeOH:ACN (1:1). The following gradient was used: 0-12 min, 5% buffer B; 13-14 min, 95% buffer B, 15-17 min, 5% buffer B. Under these conditions, ribitol eluted at 8.3 min. The metabolite was analyzed using electrospray ionization mass spectrometry operated in positive ion mode, ESI+. Compound concentrations in tissue samples were determined based on standard curves prepared by serial dilutions (200-0.01 μM) of the compound in MeOH:ACN (1:1).

### Example 3. Controlled Release Polymers for Delivery of Ribitol/Ribose for Treatment of LGMD2i (Limb Girdle Muscular Dystrophy Type 2i)

The technology relates to the controlled delivery of ribitol sugars using cross-linked polymers for the treatment of dystroglycanopathies. Ribitol is a pentose sugar, which occurs naturally as d-ribitol.

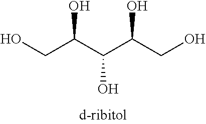

Treatment with d-ribitol has been shown to unexpectedly enhance glycosylation of alpha dystroglycan in mutant mice with dystroglycanopathies. These results, together with the favorable toxicology profile of this simple sugar, provide evidence that ribitols may provide an effective treatment for muscular dystrophies associated with dystroglycanopathies. However, a controlled release formulation is needed in order to ensure a continuous high level of ribitol in the blood-stream.

In some embodiments, the present invention provides a controlled release formulation of ribitol for treating LGMD2i (Limb Girdle Muscular Dystrophy type 2i)

We describe use of controlled release polymers to create an optimal steady state therapeutic dose of ribitol/ribose for the treatment of the genetic dystrophic disease LGMD2i. Ribitol and ribose likely play a role in muscle function by creating polyribitol cross links between muscle fibers thereby allowing muscle fibers to coordinate movement. In the genetic disease LGMD2i, mutations in the FKRP gene diminish polyribitol cross links leading to cross link breakage with consequent muscle weakness and scarring. We have shown that ribitol supplementation (po (by mouth) in drinking water) can restore normal levels of cross linking with restoration of normal muscle function.

However, because ribitol is rapidly cleared by urinary excretion it is difficult to maintain the required steady state levels of ribitol for therapy, which we have discovered are approximately 10 mg/l (serum concentration). This is roughly 1000× in excess of normal ribitol serum levels but is tolerated with no observable side effects. Maintaining such high levels for an adult human by direct ingestion of 10 wt pet ribitol solution would require ingestion of over 300 grams/day, which is therapeutically impractical.

We therefore disclose the use of controlled release polymers to maintain the required therapeutic dose for extended periods. A number of cross linked polymers optimized for delivery of water soluble polymers have been described. The current invention defines the functional characteristics of any such polymers for the specific treatment of LGMD2i via ribitol/ribose release based on our discovery in a functional animal model for LGMD2i.

In some embodiments, the polymer can be loaded to >250 mg of ribitol/L serum, with an elution rate of 5-10%/hr, resulting in an optimal steady state serum concentration at or above the therapeutic dose of 10 mg/liter, (and below 100 mg/l to insure no adverse effects) for a 6-12 hour period. This will result in a 2-4 x/day dosing schedule to optimize dosing compliance.

Ribitol is rapidly cleared by urinary excretion and therefore it is difficult to maintain the required steady state levels of ribitol for therapy, which we have discovered are about 10 mg/L (serum concentration). Ribitol and ribose given by gavage are rapidly cleared from the blood stream and the serum levels drop to near background levels within 2 hours as illustrated in Table 1.

This serum concentration is roughly 1000× in excess of normal ribitol serum levels but is tolerated with no observable side effects, and maintaining such high levels for an adult human by direct ingestion of 10 wt pct ribitol solution would require ingestion of over 300 grams/day, which would be therapeutically impractical without a controlled release formulation. Therefore maintaining an effective steady state serum level of the active component requires about 5-10 g/kg body weight daily with frequent administration.

As a nonlimiting example of a formulation of this invention, METHOCELDC2 (Hypromellose) can be mixed with the active agent in percentages of about 25% and about 50%, respectively, together with a portion of microcrystalline cellulose and the colloidal silicon dioxide. The mixture can then be blended in a suitable mixer until homogeneously mixed. Magnesium stearate can then be added and the mixture can be blended for a few minutes. The mixture can then be compressed into tablets and can include a suitable film coating to impart mechanical strength, or encapsulated to enhance appearance and product stability. This formulation ensures a release rate of about 10-15% hourly.

While there are shown and described particular embodiments of the invention, it is to be understood that the invention is not limited thereto but may be otherwise variously embodied and practiced within the scope of the following claims. Since numerous modifications and alternative embodiments of the present invention will be readily apparent to those skilled in the art, this description is to be construed as illustrative only and is for the purpose of teaching those skilled in the art the best mode for carrying out the present invention. Accordingly, all suitable modifications and equivalents may be considered to fall within the scope of the following claims.

TABLE 1
Serum ribitol measurement in normal control and 5% ribitol
(in drinking water) - treated animal animals:

| Serum sources | ribitol (ng)/ml | Mean ribitol (ug)/L |
|---|---|---|
| Normal control (n = 5) | 162, 65, 78, | 113 ug |
| | 80, 89, 91 | |
| 5% ribitol in drinking | 924, 4221, 3184, | 2355 ug |
| water (n = 4) | 1092, | |

That which is claimed is:

1. A method of treating a disorder associated with muscle weakness in a subject, comprising administering to the subject a controlled-release composition comprising an effective amount of ribitol and/or ribose, thereby treating the disorder associated with muscle weakness.

2. The method of claim 1, wherein the effective amount of ribitol and/or ribose is in a range from about 30% to about 100% of the controlled-release composition.

3. The method of claim 2, wherein the effective amount of ribitol and/or ribose is in a range from about 50% to about 80% of the controlled release composition

4. The method of claim 1, wherein the administering of the controlled release composition comprising an effective amount of ribitol and/or ribose results in a serum level in the subject of ribitol and/or ribose in a range from about 200 ug/L to about 20 mg/L.

5. The method of claim 1, wherein the administering of the controlled release composition comprising an effective amount of ribitol and/or ribose results in a serum level in the subject of ribitol and/or ribose in a range from about 0.5 mg/L to about 5 mg/L.

6. The method of claim 1, wherein the disorder associated with muscle weakness is muscular dystrophy.

7. The method of any of claim 1, wherein the disorder associated with muscle weakness is a disorder associated with a mutation or loss of function in a fukutin related protein (FKRP) gene and/or a disorder associated with a defect in glycosylation of alpha-DG in the subject.

8. The method of any of claim 1, wherein the subject is a carrier of a mutated FKRP gene with or without a defect in glycosylation of alpha-DG.

9. A method of treating or inhibiting the development of muscle weakness in a subject, comprising administering to the subject a controlled-release composition comprising an effective amount of ribitol and/or ribose, thereby treating or inhibiting the development of muscle weakness.

10. The method of claim 1, wherein the controlled-release composition is a polymer based controlled release system, a micro-capsulation based controlled release system, or an osmotic controlled release oral delivery system (OROS).

11. The method of claim 10, wherein the controlled-release composition is a polymer based controlled release system comprising a cross-linked polymer matrix loaded with an effective amount of ribitol and/or ribose.

12. The method of claim 11, wherein the cross-linked polymer matrix comprises a cellulose based polymer, a non-cellulose based polymer, a natural polymer, an acrylic acid based polymer, or any combination thereof.

13. The method of claim 11, wherein the controlled-release composition comprises hydroxypropyl methylcellulose (HMPC), methylcellulose, chitosan, hydroxyethyl methacrylate (HEMA), alginate, fibrin, gelatin, collagen, hyaluronic acid, dextran, N-(2-hydroxypropyl)methacrylate (HPMA), N-vinyl-2-pyrrolidone (NVP), N-isopropyl acrylamide (NIPAAm), vinyl acetate (VAc), acrylic acid (AA), methacrylic acid (MAA), microcrystalline cellulose (MCC), polyethylene glycol acrylate/methacrylate (PEGA/PEGMA), polyethylene glycol diacrylate/dimethacrylate (PEGDA/PEGDMA), 2-(dimethylamine)ethyl methacrylate (DMAEMA, polypropylene oxide-polyethylene oxide-polypropylene oxide (PPO-PEO-PPO) block polymers, or any combination thereof.

14. The method of claim 12, wherein the cross-linked polymer matrix comprises hydroxypropyl methylcellulose (HMPC) and microcrystalline cellulose (MCC).

15. The method of claim 1, wherein the controlled-release composition is encapsulated or compressed into a tablet.

16. The method of claim 15, wherein the encapsulated or compressed controlled-release composition is coated with a suitable film coat, erodible outer layer composition, mucoadhesive outer layer composition, or any combination thereof.

17. The method of claim 16, wherein the erodible outer layer composition comprises HMPC, ethyl cellulose, PEO, or any combination thereof.

18. The method of claim 16, wherein the mucoadhesive outer layer composition comprises a carbohydrate polymer.

19. The method of claim 1, wherein the controlled-release composition elutes a therapeutically effective amount of ribose and/or ribitol at an elution rate of about 5-20%/hr with a daily dose from about 0.05 g/Kg to about 1 g/Kg body weight.

20. The method of claim 1, wherein the controlled-release composition elutes a therapeutically effective amount of ribose and/or ribitol at an elution rate of about 5-20%/hr with a daily dose from about 0.1 g/Kg to about 0.2 g/Kg body weight.

21. The method of claim 1, wherein the therapeutically effective amount of ribitol and/or ribose elutes at a rate to obtain a steady state serum concentration that is from about 0.5 mg/L to about 20 mg/L above normal serum levels.

22. The method of claim 1, wherein the therapeutically effective amount of ribitol and/or ribose elutes at a rate to obtain a steady state serum concentration that is from about 1 mg/L to about 5 mg/L above normal serum levels.

23. The method of claim 1, wherein a single administration of the controlled-release composition provides a therapeutically effective steady state serum concentration of ribitol and/or ribose for about 2 hours to about 24 hours.

24. The method of claim 1, wherein a single administration of the controlled-release composition provides a therapeutically effective steady state serum concentration of ribitol and/or ribose for about 6 hours to about 12 hours.

25. The method of claim 1, wherein the effective amount of ribitol and/or ribose administered to the subject over 24 hours is about 0.05 g/kg to about 1 g/kg, based on the body weight of the subject.

26. The method of claim 1, wherein the effective amount of ribitol and/or ribose administered to the subject over 24 hours is about 0.1 g/kg to about 0.2 g/kg, based on the body weight of the subject.

27. The method of claim 1, wherein the controlled-release composition is administered orally.

28. The method of claim 1, wherein the controlled-release composition further comprises one or more pharmaceutically acceptable excipients, diluents, and/or carriers.

29. The method of claim 1, wherein the controlled-release composition further comprises one or more therapeutic agents.

30. The method of claim 29, wherein the one or more therapeutic agents comprise one or more gene therapeutic agents for treating and/or inhibiting the development of muscle weakness in a subject that is a carrier of a mutated FKRP gene with or without a defect in glycosylation of α-DG and/or for treating a subject having a disorder associated with a mutation or loss of function in a fukutin related protein (FKRP) gene.
